(19) United States
(12) Patent Application Publication
(10) Pub. No.: US 2026/0219281 A1
(43) Pub. Date: Jul. 30, 2026

(54) BIOMARKERS FOR ASSESSING THE EFFECT OF DRUGS IN PATHOLOGICAL DISORDERS

(71) Applicant: DermAb. io, Haifa (IL)

(72) Inventors: Miriam ORON, Jerusalem (IL); Meital PORTUGAL COHEN, Jerusalern (IL); Guy SHALOM, Yavne (IL)

(73) Assignee: DermAb. io, Haifa (IL)

(21) Appl. No.: 19/148,604

(22) PCT Filed: Jan. 17, 2024
(86) PCT No.: PCT/IL2024/050069

§ 371 Date: Jul. 16, 2025

Related U.S. Application Data
(60) Provisional application No. 63/480,158, filed on Jan. 17, 2023.
(60) Provisional application No. 63/607,017, filed on Dec. 6, 2023.

(51) Int. Cl. G01N 33/68

(52) U.S. Cl. CPC G01N 33/6881; G01N 2800/202; G01N 2800/205; G01N 2800/52

(57) ABSTRACT

The present disclosure relates to kits, substrate materials and methods for individualizing a treatment regimen for a subject with a disease and/or monitoring disease progression in the subject, the method comprising calculating probability of treatment success for one or more treatment regimens, the probability of treatment success calculation is based on expression of at least one biomarker determined in one or more skin samples collected from the subject.

### TECHNOLOGICAL FIELD

The present invention relates to precision and personalized medicine and specifically to methods and kits for determining responsiveness to treatment.

### BACKGROUND ART

References considered to be relevant as background to the presently disclosed subject matter are listed below:

International application publication No. WO05100603

Bagel, J., Wang, Y., Montgomery, III, P., Abaya, C., Andrade, E., Boyce, C., Tomich, T., Lee, B.-I., Pariser, D., Menter, A., & Dickerson, T. (2021). A Machine Learning-Based Test for Predicting Response to Psoriasis Biologics. SKIN The Journal of Cutaneous Medicine, 5(6), 621-638.

Ibrahim S F, Taft B J, Wang Y, Lee B I, Andrade E, Abaya C, Pramanick S, Mannath T, Hurley K A, Mahmood T A, Dickerson T J. Minimally Invasive Skin Transcriptome Extraction Using a Dermal Biomarker Patch. Dermatol Ther. (Heidelb). 2022 June; 12(6):1313-1323.

Acknowledgement of the above references herein is not to be inferred as meaning that these are in any way relevant to the patentability of the presently disclosed subject matter.

### BACKGROUND

Precision and personalized medicine has become an increasingly important tool in assessing an individual's or a group of individuals response to a given therapy, allowing tailored made therapy.

For example, determining responsiveness of patients diagnosed with autoimmune disease or skin disease is highly valuable as this class of disease include a large and diverse number of diseases that are treated with various therapies. Several autoimmune diseases, such as psoriasis, are known to be associated with skin disorders.

Diagnosis of skin disease and monitoring treatment efficacy in patients suffering from skin disease, including, inter alia, psoriasis was previously reported.

International application publication No. WO05100603 provides non-invasive methods for detecting, monitoring, and diagnosing skin disease and pathological skin states such as irritated skin and psoriasis using tape stripping to analyze expression in epidermal samples, of one or more skin markers.

Ibrahim S F et al showed difference in gene expression between normal skin and psoriatic lesions. Specifically, three genes were up-regulated genes (S100A9, KRT16, IL38G), and three genes were downregulated (IL37, KRT77, BTC).

Bagel J et al conducted a study that was aimed at predicting response to IL-17, TNF-α and IL-23 inhibitors using mRNA biomarkers from the epidermis and upper dermis.

### GENERAL DESCRIPTION

In accordance with some aspects, the present disclosure provides a method for individualizing a treatment regimen for a subject with a disease and/or monitoring disease progression in the subject, the method comprising calculating probability of treatment success for one or more treatment regimens, the probability of treatment success calculation is based on expression of at least one biomarker determined in one or more skin samples collected from the subject.

In accordance with some aspects, the present disclosure provides a method for individualizing a treatment regimen for a subject with a disease and/or monitoring disease progression in the subject, the method comprising calculating probability of treatment success for one or more treatment regimens, the probability of treatment success calculation is based on expression of at least one biomarker determined in one or more skin samples collected from the subject, a measure of at least one clinical feature of the subject or a combination thereof.

In accordance with some aspects, the present disclosure provides a method for assessing responsiveness to a treatment regimen for a subject suffering from a pathological disorder and monitoring disease progression of the subject. The method comprising the steps of (a) determining at least one of (i) an expression of at least one biomarker in at least one skin sample collected from the subject or in a skin area (skin region) of the subject, (ii) a measure of at least one clinical feature of the subject or (iii) a combination thereof, and (b) determining if the subject is responsive or non-responsive to the treatment regimen.

In some embodiments, the at least one skin sample is collected from the subject using non-invasive techniques. In some embodiments, the at least one skin sample is collected from the subject using a substrate material.

In accordance with some further aspects, the present disclosure provides a method for determining a personalized treatment regimen for a subject suffering from a pathological disorder, by assessing responsiveness of the subject to a treatment regimen and/or monitoring disease progression of the subject. The methods comprising the following steps: (a) determining at least one of: (i) an expression of at least one biomarker in at least one biological sample obtained from the subject or in a skin area of the subject, (ii) a measure of at least one clinical feature obtained from the subject or (iii) a combination thereof, (b) classifying the subject and (c) one or more of: (i) administering the treatment regimen to a subject classified as a responder, (ii) maintaining the treatment regimen to a subject classified as a responder, (iii) increasing the dose of the treatment in subject exhibiting a mild or poor response, or (iv) ceasing the treatment regimen for a subject classified as a non-responder or poor responder.

In accordance with some other aspects, the present disclosure provides a kit for use in determining the efficacy of a treatment regimen in a subject suffering from a pathological disease, the kit comprises (i) means to collect at least one skin sample and/or (ii) means to determine the expression of at least one biomarker in said skin sample and/or the at least one physical measure. In some examples, the kit comprises instruction for use of the kit in the methods of the present disclosure.

In accordance with some aspects, the present disclosure provides a method for collecting at least one skin sample from a subject, the method comprising contacting at least one skin region in the subject with a substrate material, wherein the substrate material exhibits inert characteristics and wherein the skin sample is suitable for characterizing at least one biomarker in the at least one skin sample of the subject.

In accordance with some other aspects, the present disclosure provides a method for collecting at least one skin sample from a subject, the method comprising contacting at least one skin region in the subject with a substrate material, wherein the substrate material is essentially free of oxidation and/or reduction characteristics and wherein the skin sample is suitable for characterizing at least one biomarker in the at least one skin sample of the subject.

In accordance with some further aspects, the present disclosure provides a method for collecting at least one skin sample from a subject, the method comprising contacting at least one skin region in the subject with a substrate material, wherein the substrate material is essentially free of an oxidizing agent and/or essentially free of an antioxidant agent and wherein the skin sample is suitable for characterizing at least one biomarker in the at least one skin sample of the subject.

In accordance with some other aspects, the present disclosure provides a method for diagnosis or detecting a pathological disorder in a subject, the method comprises determining an expression of at least one biomarker in at least one skin sample collected from the subject, and determining if the subject suffers from the disorder, wherein the skin sample is collected by a method comprising contacting at least one skin region in the subject with a substrate material or a part thereof, wherein the substrate material is essentially free of oxidation and/or reduction characteristics and wherein the skin sample is suitable for characterizing at least one biomarker in the at least one skin sample of the subject.

In accordance with some further aspects, it is provided a method for diagnosis or detecting a pathological disorder in a subject, the method comprises (a) determining an expression of at least one biomarker in at least one skin sample collected from the subject and (b) determining if the expression value obtained in (a) for each biomarker is positive or negative with respect to a predetermined standard expression value or to an expression value of the biomarker in at least one control sample, wherein the skin sample is collected by a method comprising contacting at least one skin region in the subject with a substrate material or a part thereof, wherein the substrate material is essentially free of oxidation and/or reduction characteristics and wherein the skin sample is suitable for characterizing at least one biomarker in the at least one skin sample of the subject.

In accordance with yet some aspects, the present disclosure provides a method for assessing responsiveness to a treatment regimen for a subject suffering from a pathological disorder and monitoring disease progression of the subject, the method comprising determining an expression of at least one biomarker in at least one skin sample collected from the subject, and determining if the subject is responsive or non-responsive to the treatment regimen, wherein the skin sample is collected by a method comprising contacting at least one skin region in the subject with a substrate material or a part thereof, wherein the substrate material is essentially free of oxidation and/or reduction characteristics and wherein the skin sample is suitable for characterizing at least one biomarker in the at least one skin sample of the subject.

In accordance with yet some other aspects, the present disclosure provides a method for assessing responsiveness to a treatment regimen for a subject suffering from a pathological disorder and monitoring disease progression of the subject, the method comprises (a) determining an expression of at least one biomarker in at least one skin sample collected from the subject and (b) determining if the expression value obtained in (a) for each biomarker is positive or negative with respect to a predetermined standard expression value or to an expression value of the biomarker in at least one control sample, wherein the skin sample is collected by a method comprising contacting at least one skin region in the subject with a substrate material or a part thereof, wherein the substrate material is essentially free of oxidation and/or reduction characteristics and wherein the skin sample is suitable for characterizing at least one biomarker in the at least one skin sample of the subject.

In accordance with yet some further aspects, the present disclosure provides a substate material being essentially free of oxidation and/or reduction characteristics for use in a method of collecting at least one skin sample from a subject.

In accordance with yet some other aspects, the present disclosure provides a substate material being essentially free of oxidation and/or reduction characteristics for use in a method for collecting at least one skin sample from a subject, the method comprising contacting at least one skin region in the subject with the substrate material, wherein the skin sample is suitable for characterizing at least one biomarker in the at least one skin sample of the subject.

In accordance with yet some other aspects, the present disclosure provides a kit comprising a substate material and instruction for use of the substate material in a method of collecting at least one skin sample from a subject, wherein the substrate material is essentially free of oxidation and/or reduction characteristics for and the method comprises contacting at least one skin region in the subject with the substrate material and optionally characterizing at least one biomarker in the at least one skin sample of the subject.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic representation of a model providing likelihood of success for some treatment regimens considered for a subject.

FIG. 2 is a graph showing oxygen radical absorbance capacity (ORAC) measurements in skin areas of different healthy individuals (total of 4 individuals) using non-sterile cotton Q-tips swabs (same as cotton buds in British English).

FIG. 3 is a graph showing ORAC measurements in skin areas using non-sterile swabs in healthy individuals at different ages, left vs. right inner forearm.

FIG. 4 is a graph showing ORAC measurements in skin areas using non-sterile swabs in healthy individuals at different ages over time.

FIG. 5 is a graph showing IL-1a measurements in skin areas using non-sterile swabs in healthy individuals at different ages, left vs. right inner forearm.

FIG. 6 is a graph showing IL-1RA measurements in skin areas using non-sterile swabs in healthy individuals, left vs. right inner forearm.

FIG. 7 is a graph showing IL-1RA/IL-1a in skin areas using non-sterile swabs in healthy individuals at different ages, left vs. right inner forearm.

FIG. 8 is a graph showing IL-1a measurements in skin areas using non-sterile swabs in healthy individuals at different ages over time.

FIG. 9 is a graph showing IL-1RA measurements in skin areas using non-sterile swabs in healthy individuals at different ages over time.

FIGS. 10A and 10B is a bar graph of cytokine levels (FIG. 10A) and ORAC levels (FIG. 10B) in responders and non-responders using skin wash samples from lesional (LE) skin tissue, * denotes p<0.05.

FIGS. 11A and 11B is a bar graph of cytokine levels (FIG. 11A) and ORAC levels (FIG. 11B) in responders and non-responders using skin wash samples from non-lesional (NL) skin tissue, * denotes p<0.05.

FIG. 12 is a graph showing Interleukin (IL-10) expression in healthy individuals and in psoriasis lesions and in non-lesional regions in psoriasis patients.

FIG. 13 is a graph showing KLK5 expression in lesional and non-lesional in skin areas using non-sterile swabs in psoriasis patients.

FIG. 14 is a graph showing albumin expression in lesional and non-lesional in skin areas using non-sterile swabs in psoriasis patients.

FIGS. 15A-15E are graphs showing expression of various biomarkers in in healthy individuals and in lesions (LE) and in non-lesional (NL) regions in atopic dermatitis (AD) patients, albumin (FIG. 15A), KLK5 (FIG. 15B), IL-1RA (FIG. 15C), IL-1a, (FIG. 15D), ORAC (FIG. 15E).

### DETAILED DESCRIPTION OF EMBODIMENTS

The present application is based on identification of biomarkers that can be used in diagnosis of disease and in assessing response to therapy in subjects suffering from various pathological conditions, including conditions that are manifested in the skin of a subject.

Specifically, and as shown in the Examples below, the inventors identified several biomarkers, including, inter alia, cytokines and antioxidants, that their expression prior to any treatment (i.e. at baseline expression) was found to correlate with responsiveness to a treatment.

It was suggested that the biomarkers identified herein may be used to assess responsiveness to treatment and to monitor disease progression and importantly may be used for personalized or precision medicine.

This may allow healthcare providers to tailor treatment plans based on an individual's unique characteristics, potentially leading to more effective and targeted interventions.

In addition, and as shown in the examples below, the inventors identified specific types of materials including, inter alia, inert materials (sterile and non-sterile) that may be used for accurately determine expression of these biomarkers in biological samples and/or determine antioxidant potential of these samples. As shown below, expression of biomarkers and antioxidant potential of skin samples can be determined using inert materials, such as inert cotton-based materials, while other materials such as medical cotton-based non-inert swabs have a high background measurement and hence can not be used to accurately determine these parameters.

Since accurate and reproducible determination of biomarkers and/or antioxidant potential of skin samples is highly valuable, it was suggested that the materials described herein, may be used in non-invasive methods to determine one or more skin biomarkers, including antioxidant potential of skin samples, information that may be used as diagnostic tools and/or as prognostic tool, ultimately allowing early diagnosis of skin conditions as well as in predicting response to treatment.

In accordance with some aspects, the present disclosure provides a method for determining a personalized treatment regimen for a subject suffering from a pathological disease by assessing responsiveness of the subject to a treatment regimen and/or monitoring disease progression of the subject.

It was suggested that the identified biomarkers may be combined with one or more physiological parameters obtained from the subject in order to predict responsiveness to treatment in the subjects.

Hence, the present disclosure provides a method for personalizing a treatment regimen for a subject with a disease, comprising classifying the subject as a responder or a non-responder to the treatment regimen, the classification is based on at least one of (i) expression of at least one biomarker determined in one or more biological samples collected from the subject, (ii) a measure of at least one clinical feature obtained from the subject or (iii) a combination thereof.

As described herein, the methods allow individualized and targeted selection of treatments, potentially leading to better outcomes and reduced adverse effects. This may be achieved by using individual's own information, for example, biomarkers expression, determined at any time point prior to treatment initiation and providing a probability of treatment success or recommendation for effectiveness of treatment options.

Hence, the present disclosure is directed to method for individualizing a treatment regimen for a subject with a disease, the method comprising calculating probability of treatment success for one or more treatment regimens, the probability of treatment success calculation is based on expression of at least one biomarker determined in one or more biological samples collected from the subject.

Based on the probability of treatment success, an informed decision regarding the most suitable treatment can be made both in terms of effectiveness and side effects.

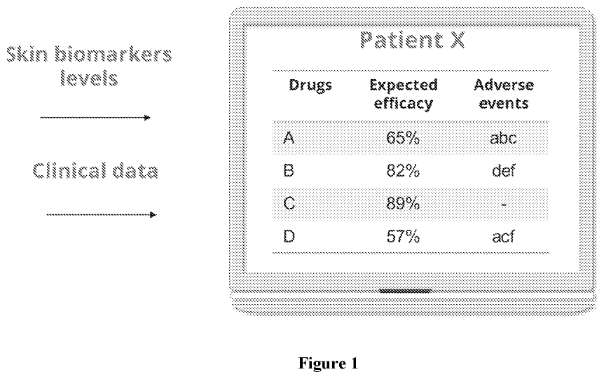

A schematic representation of the likelihood of success for some treatment regimens considered for a subject is shown in FIG. 1. The schematic representation shows expected efficacy and adverse effects such that based on these parameters, a specific treatment regimen (e.g. a drug) is ultimately selected.

In accordance with some embodiments that may be considered as aspects of the present disclosure, the method comprises selecting a treatment regimen for the subject based on the probability of treatment success calculated for one or more treatment regimens.

In accordance with some embodiments that may be considered as aspects of the present disclosure, the method comprising selecting a treatment regimen for the subject from one or more treatment regimens, the selection is based on probability of treatment success for the one or more treatment regimens calculated based on expression of at least one biomarker determined in one or more biological samples collected from said subject.

In accordance with some embodiments that may be considered as aspects of the present disclosure, the method comprising calculating probability of treatment success for one or more treatment regimens and selecting a treatment regimen for the subject from the one or more treatment regimens, the probability of treatment success calculation is based on expression of at least one biomarker determined in one or more biological samples collected from said subject.

The individualization and targeted selection of treatments that may be implemented by determining if a subject is classified as responder or non-responder to a specific treatment may be done by any computational means.

In some examples, classification is based on probability of treatment success for the one or more treatment regimens.

The probability of treatment success can be calculated for example using the following approaches.

In some examples, the probability (likelihood) of treatment success may be calculated by the Subset Selection approach. This approach identifies a subset of predictors that are able to predict the response to therapy or diseases severity. A model is then fitted using linear and non-linear regression on the reduced set of variables. All the possible combinations of variables are explored, starting from a single variables and combinations of the others, until the full set is used. For a certain number of predictors (variables) the best model is selected by using an appropriate indicator, e.g., Residual Sum of Squares (RSS) or adjusted R², Mallow's Cp indicator, Bayesian Information Criterion (BIC).

In some examples, the probability (likelihood) of treatment success may be calculated by machine learning (ML) algorithm, Support Vector Machine (SVM) such as Kernel functions and Neural network (NN) algorithm. Such algorithms are used to train an algorithm that predicts response to therapy, diseases severity or patients' profiles, based on variables data sets. Iterations between test and training tests are performed until a satisfactory result is achieved and considered valid. The most significant features for the prediction are also identified in this analysis.

As appreciated, such mathematical models may be formed by collecting comprehensive data on the identified biomarkers from individuals who have undergone different treatments. This dataset should include information about the biomarker levels and the corresponding treatment outcomes (e.g. responder or non-responder). The model may be developed based on a statistical or a machine learning model.

As an outcome, the model be a predictive model that can indicate the likelihood (probability) of treatment success (responsiveness) or failure (non-responsiveness) based on specific biomarker patterns for one or more treatment regimens.

Hence, the present disclosure provides a method for personalizing a treatment regimen for a subject with a disease, comprising classifying the subject as a responder or a non-responder to the treatment regimen, the classification is based on expression of at least one biomarker determined in one or more biological samples collected from the subject.

In some aspects, the method comprises a step of receiving information from one or more skin sample collected from the subject, the information may include information on expression of one or more biomarkers in the one or more skin samples and/or on measures related to at least one clinical feature. As described herein, this information is used for individualizing a treatment regimen for the subject by determining responsiveness and/or non-responsiveness to a specific treatment regimen.

In some aspects, the method comprises receiving expression of one or more biomarkers determined in one or more skin samples collected from a subject and calculating probability of treatment success for one or more treatment regimens based on the expression of the one or more biomarkers.

In some other aspects, the method comprises receiving one or more measures of at least one clinical feature obtained from a subject and calculating probability of treatment success for one or more treatment regimens based on the measure of the at least one clinical feature.

In some further aspects, the method comprises receiving one or more of (i) expression of one or more biomarkers determined in one or more skin samples collected from a subject, (ii) one or more measures of at least one clinical feature obtained from a subject or (iii) a combination thereof and calculating probability of treatment success for one or more treatment regimens based on the expression, the measure or a combination thereof.

The methods comprise determining expression of one or more biomarkers such that this expression is received and used for adjusting (individualizing) treatment regimen.

In some embodiments that may be considered as aspects of the present disclosure, the method comprises determining an expression of at least one biomarker in at least one skin sample collected from the subject or in a skin area of the subject.

In some embodiments that may be considered as aspects of the present disclosure, the method comprises determining a measure of at least one clinical feature obtained from the subject.

In some embodiments that may be considered as aspects of the present disclosure, the method comprises determining at least one of: (i) an expression of at least one biomarker in at least one biological sample collected from the subject or in a skin area of the subject, (ii) a measure of at least one clinical feature obtained from the subject or (iii) a combination thereof.

In some embodiments that may be considered as aspects of the present disclosure, the comprising determining expression of one or more biomarkers from the subject and calculating probability of treatment success for one or more treatment regimens based on the expression of the one or more biomarkers.

In some embodiments that may be considered as aspects of the present disclosure, the comprising determining expression of one or more biomarkers from the subject, calculating probability of treatment success for one or more treatment regimens based on the expression of the one or more biomarkers, and selecting a treatment regimen for the subject from the one or more treatment regimen.

In some examples, the method comprises (a) determining an expression of at least one biomarker in at least one skin sample collected from the subject or in a skin area of the subject and (b) classifying the subject.

In some examples, the method comprises (a) determining a measure of at least one clinical feature obtained from the subject and (b) classifying the subject.

In accordance with some other aspects, the present disclosure provides a method for determining a personalized treatment regimen for a subject suffering from a pathological disorder by assessing responsiveness of the subject to a treatment regimen and/or monitoring disease progression of the subject. The methods comprising the following steps: (a) determining at least one of: (i) an expression of at least one biomarker in at least one biological sample collected from the subject or in a skin area of the subject, (ii) a measure of at least one clinical feature obtained from the subject or (iii) a combination thereof and (b) classifying the subject.

In the following text, when referring to a method it is to be understood as also referring to the composition, kits or materials disclosed herein. Thus, whenever providing a feature with reference to the methods, it is to be understood as defining the same feature with respect to the composition, kits and materials, mutatis mutandis.

In some examples, the method comprises classifying the subject as a responder or a non-responder to one or more treatment regimens, the classification is based on expression of at least one biomarker determined in one or more biological samples collected from the subject. In some examples, the classification is based on probability of treatment success for the one or more treatment regimens.

In some examples, the method comprising receiving expression of one or more biomarkers determined in one or more biological samples collected from the subject and calculating probability of treatment success for one or more treatment regimens based on the expression of the one or more biomarkers.

In some examples, the method comprising receiving expression of one or more biomarkers determined in one or more biological samples collected from the subject and classifying the subject as a responder or a non-responder to one or more treatment regimens based on expression of the one or more biomarkers.

In some embodiments, the method comprises an additional step comprising one or more of: (i) administering the treatment regimen to a subject classified as a responder, (ii) maintaining the treatment regimen to a subject classified as a responder, or (iii) increasing the dose of the treatment in subject exhibiting a mild or poor response. As described above, the step of administering the treatment regimen to a subject classified as a responder is a result of selecting an appropriate/suitable treatment regimen.

In some embodiments, the method comprises an additional step comprising administering the treatment regimen to a subject classified as a responder.

In accordance with some embodiments, the method comprises collecting one or more biological samples from the subject.

In accordance with some embodiments, the method comprises collecting one or more biological samples from the subject using a non-invasive method (technique).

The term “biological sample” as used in the present disclosure is meant to include samples obtained from a mammal subject.

It should be note that a biological sample may be for example, skin, bone marrow, lymph fluid, blood cells, blood, serum, plasma, urine, sputum, saliva, faeces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents.

In some embodiments, the sample may be a skin sample.

In some embodiments, the method of the invention is applicable for a skin sample.

In accordance with some embodiments, the method comprises collecting one or more skin samples from the subject.

In accordance with some embodiments, the method comprises collecting one or more skin samples from the subject using a non-invasive method (technique), for example, from the surface of a skin tissue of a subject.

In accordance with some embodiments, the method comprises collecting at least one skin sample from at least one skin are prior to initiation of treatment.

In some embodiments, the method of the invention is applicable for a skin area of the subject.

The skin sample can be collected from various regions in the body.

The skin sample can be collected from the trunk. The trunk refers to a body that contains the chest, abdomen, pelvis, and back.

In some embodiments, the method comprises collecting a skin sample from a skin area being an elbow, a knee, an elbow a wrist, a lower back area, a scalp, a trunk or any combination thereof.

In some embodiments, the method comprises collecting a skin sample from a skin area being an inner part of a body region. In some embodiments, the method comprises collecting a skin sample from a skin area being an inner part of an elbow, an inner part of a knee, an inner part of a wrist, or any combination thereof.

In some embodiments, the method comprises collecting a skin sample from a skin area being an outer part of a body region. In some embodiments, the method comprises collecting a skin sample from a skin area being an outer part of an elbow, an outer part of a knee, an outer part of a wrist, or any combination thereof.

As appreciated, various pathological disorders are associated with intrinsic mechanisms that cause changes in the skin homeostasis, such changes may at times be visible in the skin tissue (denoted herein lesional skin) and at times invisible in the skin tissue (denoted herein as non-lesional skin).

In some embodiments, the method comprises collecting a skin sample from a skin area being a lesional skin area. In some embodiments, the method comprises collecting a skin sample from a lesional skin.

In some embodiments, the method comprises collecting a skin sample from a lesional skin area of a subject suffering from a pathological disorder.

As used herein the term lesional area or lesional skin tissue refers to any skin area that has different features relative to a control skin tissue (e.g. of a healthy subject), including, inter alia, different color, different shape, different size, and different texture, different temperature.

In some embodiments, the lesion skin tissue is characterized by one or more of redness, plaque, scaly skin.

In some embodiments, the lesion skin tissue is characterized by a skin plaque. A skin plaque as used herein refers to an elevated, solid, superficial lesion, optionally being larger than 10 mm in diameter.

In some embodiments, the method comprises collecting a skin sample from a skin area being a non-lesional skin area. In some embodiments, the method comprises collecting a skin sample from a non-lesional skin area.

In some embodiments, the method comprises collecting a skin sample from a non-lesional skin area of a subject suffering from a pathological disorder.

As changes in non-lesional skin are invisible and non-lesional skin could become lesional, monitoring skin area, for example by collecting skin sample from non-lesional areas is important.

The non-lesional skin in the context of the present disclosure is a skin tissue obtained from a subject suffering from a pathological disorder that has no significant changes in the visual appearance.

A non-lesioned skin can be characterized by at least one of a symmetric shape, absence of irregular structures, not scaled, without having changes in color or any combination thereof.

In some embodiments, the lesional area and the non-lesional area are in the same body region. In some embodiments, the lesional area and the non-lesional area are in different body regions.

In some embodiments, the distance between lesional area and non-lesional area is at least about 1 cm, at times at least about 2 cm, at times at least about 5 cm, at times at least about 10 cm.

In some embodiments, the distance between lesional area and non-lesional area is between about 1 cm and about 10 cm, at times about 1 cm and about 5 cm, at times about 1 cm and about 4 cm.

The skin sample described herein, either from a lesional area, a non-lesional area or both can be collected from one or more of the skin layers, including, inter alia, the epidermis, dermis, and the hypodermis.

As known in the art, epidermis refers to the outmost thinnest layer of skin composed of five layers, including, stratum corneum, stratum lucidum, stratum granulosum, stratum spinosum and stratum basale.

In some embodiments, the method comprises collecting a skin sample from the epidermis.

In some embodiments, the method comprises collecting a skin sample the skin surface.

In some embodiments, the method comprises collecting a skin sample from the epidermis surface.

In some embodiments, the method comprises collecting a skin sample from the stratum corneum.

As appreciated, the skin sample include one or more biomarkers which expression is determined in the methods of the invention.

In some embodiments, the skin sample comprises at least one biomarker from the epidermis.

In some embodiments, the skin sample comprises at least one biomarker secreted from the epidermis.

In some embodiments, the skin sample comprises at least one biomarker from the surface of the epidermis.

In some embodiments, the skin sample comprises at least one biomarker secreted from the surface of the epidermis.

In some embodiments, the skin sample comprises at least one biomarker from the surface of the stratum corneum.

In some embodiments, the skin sample comprises at least one biomarker secreted from the surface of the stratum corneum.

It should be noted that when referring to biomarkers secreted from the epidermis, the surface of the epidermis, the surface of the stratum corneum or the like, it encompasses biomarkers that may be produced in any one of the skin tissue layers (dermis or hypodermis) and may reach the epidermis, the surface of the epidermis, the surface of the stratum corneum to be secreted therein.

As described herein, the method of the invention relates to determining expression of at least one skin biomarker in a skin area and/or skin sample.

The present disclosure is relevant for various biomarkers and encompasses hydrophilic biomarkers and/or hydrophobic biomarkers.

In some embodiments, the at least one biomarker is at least one hydrophilic biomarker.

In some embodiments, the at least one biomarker is at least one hydrophobic biomarker. The hydrophobic biomarker is or comprises in accordance with some embodiments, a lipid moiety. In some embodiments, the lipid molecule is at least one of a fat, a wax, a sterol, a fat-soluble vitamin, monoglyceride, diglyceride, phospholipid or any combination thereof. In some embodiments, the hydrophobic biomarker is a wax molecule. In some embodiments, the wax molecule is or comprises a ceramide,

In some embodiments, the at least one biomarker is at least one protein, a peptide, an antioxidant, a metabolite, an enzyme or any combination thereof.

In some embodiments, the at least one biomarker is at least one protein.

In some embodiments, the at least one protein is at least one cytokine.

As appropriated, the term cytokine refers to a family of small proteins (peptides) that are important in cell signaling.

The cytokine family can be classified into lymphokines, interleukins, and chemokines.

In some embodiments, the at least one biomarker is at least one cytokine from the interleukin family.

In some embodiments, the at least one biomarker is at least one cytokine from (i) one or more cytokine from Interleukin-1 (IL-1) family, (ii) one or more cytokine from IL-2 family, (iii) one or more cytokine from IL-3 family, (iv) one or more cytokine from IL-4 family, (v) one or more cytokine from IL-5 family, (vi) one or more cytokine from IL-6 family, (vii) one or more cytokine from IL-7 family, (viii) one or more cytokine from IL-8 family, (ix) one or more cytokine from IL-9 family, (x) one or more cytokine from IL-10 family, (xi) one or more cytokine from IL-11 family, (xii) one or more cytokine from IL-12 family, (xiii) one or more cytokine from IL-13 family, (xiv) one or more cytokine from IL-15 family, (xv) one or more cytokine from IL-17 family or (xvi) a combination thereof.

In some embodiments, the at least one biomarker is at least one cytokine from the Interleukin-1 (IL-1) family. In some embodiments, the at least one biomarker is at least one cytokine from the IL-2 family. In some embodiments, the at least one biomarker is at least one cytokine from the IL-3 family. In some embodiments, the at least one biomarker is at least one cytokine from the IL-4 family. In some embodiments, the at least one biomarker is at least one cytokine from the IL-5 family. In some embodiments, the at least one biomarker is at least one cytokine from the IL-6 family. In some embodiments, the at least one biomarker is at least one cytokine from the IL-7 family. In some embodiments, the at least one biomarker is at least one cytokine from the IL-8 family. In some embodiments, the at least one biomarker is at least one cytokine from the IL-9 family. In some embodiments, the at least one biomarker is at least one cytokine from the IL-10 family. In some embodiments, the at least one biomarker is at least one cytokine from the IL-11 family. In some embodiments, the at least one biomarker is at least one cytokine from the IL-12 family. In some embodiments, the at least one biomarker is at least one cytokine from the IL-13 family. In some embodiments, the at least one biomarker is at least one cytokine from the IL-15 family. In some embodiments, the at least one biomarker is at least one cytokine from the IL-17 family.

In some embodiments, the at least one biomarker is one or more of IL-1α, IL-1β, IL-1α receptor antagonist (RA), IL-18, IL-33, IL-36α, IL-36β, IL-36γ, IL-37, IL-38 or a combination thereof. In some embodiments, the at least one biomarker is at least two, at least three, at least four, at least five, of IL-1α, IL-1β, IL-1α receptor antagonist (RA), IL-18, IL-33, IL-36α, IL-36β, IL-36γ, IL-37, IL-38 or a combination thereof.

In some embodiments, the at least one biomarker is at least one of IL-1α, IL-1β, IL-1RA or any combination thereof.

In some embodiments, the at least one biomarker is at least one of IL-1α, IL-1β, or any combination thereof.

In some embodiments, the at least one biomarker is one or more of IL-2, IL-7, IL-9, IL-15, IL-21 or any combination thereof.

In some embodiments, the at least one biomarker is IL-3.

In some embodiments, the at least one biomarker is IL-4, IL-5, IL-13 or a combination thereof.

In some embodiments, the at least one biomarker is IL-4, IL-5, or a combination thereof.

In some embodiments, the at least one biomarker is IL-6, IL-11, IL-27, IL-31 or a combination thereof.

In some embodiments, the at least one biomarker is IL-31.

In some embodiments, the at least one biomarker is IL-10, IL-19, IL-20, IL-22, IL-24, IL-26 or a combination thereof.

In some embodiments, the at least one biomarker is IL-22.

In some embodiments, the at least one biomarker is IL-12, IL-13, or a combination thereof.

In some embodiments, the at least one biomarker is IL-17A, IL-17B, IL-17C, IL-17D, IL-17E, IL-79F or a combination thereof.

In some embodiments, the at least one biomarker is IL-15.

In some embodiments, the at least one biomarker is IL-19, IL-20, IL-24, or a combination thereof.

In some embodiments, the at least one biomarker is one or more of IL-1α, IL-1β, IL-1α RA, IL-18, IL-33, IL-36α, IL-36β, IL-36γ, IL-37, IL-38, IL-7, IL-9, IL-15, IL-21, IL-3, IL-4, IL-5, IL-13, IL-6, IL-11, IL-27, IL-31, IL-10, IL-19, IL-20, IL-22, IL-24, IL-26, IL-12, IL-13, IL-17A, IL-17B, IL-17C, IL-17D, IL-17E, IL-79F, IL-15, IL-19, IL-20, IL-24, or a combination thereof.

In some embodiments, the at least one biomarker is IL-10.

In some embodiments, the at least one biomarker is IL-5.

In some embodiments, the at least one biomarker is IL-31.

In some embodiments, the at least one biomarker is IL-22.

In some embodiments, the at least one biomarker is IL-18.

In some embodiments, the at least one biomarker is one or more of IL-1α, IL-1β, IL-1α RA, IL-4, IL-5, IL-31, IL-22, IL-10, or a combination thereof.

In some embodiments, the at least one biomarker is one or more of IL-1α, IL-1β, IL-4, IL-31, or a combination thereof.

In some embodiments, the at least one biomarker is one or more of IL-1α RA, IL-22, IL-31 or a combination thereof.

In some embodiments, the at least one biomarker is Thymic stromal lymphopoietin (TSLP).

In some embodiments, the at least one biomarker is at least one cytokine from the chemokine family.

As appreciated, chemokine or chemotactic cytokines, refer to a family of small cytokines or signaling proteins secreted by cells that are responsible for various activities in cells, for example, inducing directional movement of leukocytes, as well as other cell types, including endothelial and epithelial cells.

In some embodiments, the at least one biomarker is at least one chemokine from the (i) CC chemokine (or β-chemokine) family, (ii) CXC chemokines (or α-chemokines), (iii) C chemokines (or γ chemokines), (iv) CX3C chemokine (or d-chemokines) or (v) any combination thereof.

In some embodiments, the at least one biomarker is at least one chemokine from the CC chemokine family.

In some embodiments, the at least one biomarker is at least one chemokine from the CXC chemokines family.

In some embodiments, the at least one chemokine from the CXC chemokines family is at least one of macrophage inflammatory protein 2 (MIP2) or IL-8.

In some embodiments, the at least one chemokine is at least one of MIP2α (also denoted as CXCL2), MIP2β (also denoted as CXCL3) or any combination thereof.

In some embodiments, the at least one chemokine is IL-8, CXCL2 or a combination thereof.

In some embodiments, the at least one chemokine is IL-8 (also denoted as CXCL8).

In some embodiments, the at least one chemokine is at least one of MIP2α (also denoted as CXCL2).

In some embodiments, the at least one biomarker is at least one chemokine from the C chemokines family.

In some embodiments, the at least one biomarker is at least one chemokine from the CX3C chemokine family.

In some embodiments, the at least one biomarker is a peptide or any fragment thereof. In some embodiments, the biomarker is or comprises an amino acid. In some embodiments, the biomarker is or comprises a tryptophan amino acid.

In some embodiments, the at least one biomarker is an antimicrobial peptide.

As appreciated, an antimicrobial peptide (AMP), also denoted as host defence peptides (HDPs) belong to the innate immune response found among all classes of life.

In some embodiments, the at least one biomarker being an antimicrobial peptide is at least one of beta defensing 2 (hBD-1), beta defensing 2 (hBD-2), beta defensing 3 (hBD-3) or any combination thereof.

In some embodiments, the at least one biomarker is a at least one kallikrein related peptide.

As appreciated kallikrein related to a group of serine proteases, enzymes capable of cleaving peptide bonds in proteins.

In some embodiments, the at least one kallikrein related peptide is at least one of KLK1, KLK2, KLK3, KLK4, KLK5, KLK6, KLK7, KLK8, KLK9, KLK10, KLK11, KLK12, KLK13, KLK14, KLK15 or any combination thereof.

In some embodiments, the at least one kallikrein related peptide is KLK5.

In some embodiments, the at least one biomarker is an enzyme.

In some embodiments, the at least one enzyme is a at least one metalloproteinase, or metalloprotease. Metalloproteinase refers to a protease enzyme whose catalytic mechanism involves a metal.

In some embodiments, the at least one biomarker being at least one metalloproteinase is at least one exopeptidase (also denoted as metalloexopeptidase).

In some embodiments, the at least one biomarker being at least one metalloproteinase is at least one endopeptidase (also denoted as metalloendopeptidase).

In some embodiments, the at least one biomarker is a at least one matrix metallopeptidase (MMP).

Matrix metalloproteinases (MMPs), also denoted as matrix metallopeptidases or matrixins, are a class of metalloproteinases that are calcium-dependent zinc-containing endopeptidases.

In some embodiments, the at least one MMP is at least one of MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-24, MMP-25, MMP-26, MMP-27, MMP-28 or any combination thereof.

In some embodiments, the at least one MMP is at least one of MMP-1, MMP-3, MMP-9 or any combination thereof.

In some embodiments, the at least one MMP is MMP-9.

In some embodiments, the at least one biomarker is a growth factor.

In some embodiments, the at least one biomarker is a Fibroblast growth factor (FGF 2).

In some embodiments, the at least one biomarker is an albumin protein.

In some examples, the at least one biomarker is one or more of IL-1α, IL-1β, IL-1α RA, IL-18, IL-33, IL-36α, IL-36β, IL-36γ, IL-37, IL-38, IL-7, IL-9, IL-15, IL-21, IL-3, IL-4, IL-5, IL-13, IL-6, IL-11, IL-27, IL-31, IL-10, IL-19, IL-20, IL-22, IL-24, IL-26, IL-12, IL-13, IL-17A, IL-17B, IL-17C, IL-17D, IL-17E, IL-79F, IL-15, IL-19, IL-20, IL-24, TSLP, IL-8, CXCL2, KLK1, KLK2, KLK3, KLK4, KLK5, KLK6, KLK7, KLK8, KLK9, KLK10, KLK11, KLK12, KLK13, KLK14, KLK15, MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-24, MMP-25, MMP-26, MMP-27, MMP-28, FGF 2, albumin or a combination thereof.

In some examples, the at least one biomarker is one or more of IL-1α, IL-1β, IL-1α RA, IL-4, IL-5, IL-31, IL-10, IL-22, TSLP, IL-8, CXCL2, KLK5, MMP-9, FGF 2, albumin or a combination thereof.

In some examples, the at least one biomarker is one or more of IL-1α, IL-1β, IL-4, IL-31, TSLP, IL-8, CXCL2, KLK5, MMP-9, FGF 2, albumin or a combination thereof.

In some embodiments, the at least one biomarker is an antioxidant. As used herein the term antioxidant refers to a compound that inhibit/reduce oxidation and hence inhibit/reduce production of free radicals.

In some embodiments, the antioxidant is a hydrophilic antioxidant, a lipophilic antioxidant, or any combination thereof. In some embodiments, the antioxidant is a lipophilic (hydrophobic) antioxidant. In some embodiments, the hydrophilic antioxidant is at least one of vitamin C, glutathione, uric acid or any combination thereof.

In some embodiments, the antioxidant is a lipophilic antioxidant.

In some embodiments, the at least one antioxidant is a dietary antioxidant.

In some embodiments, the at least one dietary antioxidant is at least one vitamin.

In some embodiments, the at least one dietary antioxidant being at least one vitamin, being vitamin E.

In some embodiments, the at least one biomarker is a metabolite. In some embodiments, the at least one biomarker being an antioxidant is a metabolite.

In some embodiments, the at least one biomarker is a nucleoside derivative.

In some embodiments, the at least one biomarker is a nucleoside breakdown product.

In some embodiments, the at least one biomarker is a purine derivative.

In some embodiments, the at least one biomarker is uric acid (UA).

As used herein, the tern urine acid refers to an antioxidant oxypurine produced from xanthine by the enzyme xanthine oxidase, and is an intermediate product of purine metabolism.

In some embodiments, the at least one biomarker is oxygen radical antioxidant capacity (ORAC).

ORAC as used herein refers to a measure of antioxidant capacity in a sample.

As can be seen in the examples below and as described above, ORAC measurements of skin samples using medical swab were high and similar to ORAC control measurements. Hence, based on these results, it was concluded that there is a noise background and ORAC assay cannot be performed using commercial medical swabs. In contrast, ORAC measurements in skin areas using non-sterile Q-tips swabs showed low and reproducible values. Taken together, these results demonstrate the ability of using inert substrate materials for accurate determination of oxidation activity/capacity of skin samples.

In some embodiments, the at least one biomarker is an antioxidant being glutathione (GSH).

GSH as used herein refers to a cysteine-containing peptide found in most forms of aerobic life. It has antioxidant properties since the thiol group in its cysteine moiety is a reducing agent and can be reversibly oxidized and reduced.

In some embodiments, the at least one biomarker is an antioxidant is one or more of UA, ORAC, GSH or a combination thereof.

In some embodiments, the protein is a fibrous protein.

A fibrous protein also denoted as scleroprotein refers to a type of a protein structure. A fibrous protein is made up of elongated or fibrous polypeptide chain that form filamentous and sheet-like structures. In some embodiments, the fibrous protein is at least one of a keratin protein, a collagen protein, an elastin protein, a fibrin protein or any combination thereof.

In some embodiments, the protein is a keratin protein.

In some examples, the at least one biomarker is one or more of IL-1α, IL-1β, IL-1α RA, IL-18, IL-33, IL-36α, IL-36β, IL-36γ, IL-37, IL-38, IL-7, IL-9, IL-15, IL-21, IL-3, IL-4, IL-5, IL-13, IL-6, IL-11, IL-27, IL-31, IL-10, IL-19, IL-20, IL-22, IL-24, IL-26, IL-12, IL-13, IL-17A, IL-17B, IL-17C, IL-17D, IL-17E, IL-79F, IL-15, IL-19, IL-20, IL-24, TSLP, IL-8, CXCL2, KLK1, KLK2, KLK3, KLK4, KLK5, KLK6, KLK7, KLK8, KLK9, KLK10, KLK11, KLK12, KLK13, KLK14, KLK15, MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23, MMP-24, MMP-25, MMP-26, MMP-27, MMP-28, FGF 2, albumin, UA, GSH, ORAC or a combination thereof.

In some examples, the at least one biomarker is one or more of IL-1α, IL-1β, IL-1α RA, IL-4, IL-5, IL-31, IL-10, TSLP, IL-8, CXCL2, KLK5, MMP-9, FGF 2, albumin, ORAC or a combination thereof.

In some examples, the at least one biomarker is one or more of IL-1α, IL-1β, IL-4, IL-31, TSLP, IL-8, CXCL2, KLK5, MMP-9, FGF 2, albumin or any combination thereof.

In some embodiments, the at least one biomarker is at least one cytokine and at least one purine derivative.

In some embodiments, the at least one biomarker is ORAC.

In some examples, the at least one biomarker is one or more of IL-1α, IL-1RA, IL-10, Albumin, KLK5, IL-4, IL-5, IL-22, IL-33, TLSP, MMP-9, IL-8, CXCL2, IL-1b, FGF basic, ORAC or a combination thereof.

In some examples, the at least one biomarker is one or more of IL-1α, IL-8, IL-1b, ORAC or a combination thereof.

In some examples, the at least one biomarker is one or more of IL-1α, IL-8, IL-1b, or a combination thereof.

In some examples, the at least one biomarker is IL-1α. In some examples, the at least one biomarker is IL-1RA. In some examples, the at least one biomarker is IL-10. In some examples, the at least one biomarker is Albumin. In some examples, the at least one biomarker is KLK5. In some examples, the at least one biomarker is IL-4. In some examples, the at least one biomarker is IL-5. In some examples, the at least one biomarker is IL-22. In some examples, the at least one biomarker is IL-31. In some examples, the at least one biomarker is TLSP. In some examples, the at least one biomarker is MMP-9. In some examples, the at least one biomarker is IL-8. In some examples, the at least one biomarker is CXCL2. In some examples, the at least one biomarker is IL-1b. In some examples, the at least one biomarker is FGF basic. In some examples, the at least one biomarker is ORAC.

In accordance with some embodiments, the methods of the invention comprise a step of collecting one or more skin samples.

In some embodiments, the methods of the invention comprise collecting one or more skin samples from a lesional area and/or a non-lesional area. In some embodiments, the methods of the invention comprise collecting one or more skin samples from a lesional area. In some embodiments, the methods of the invention comprise collecting one or more skin samples from a non-lesional area.

The skin sample can be collected by any method known in the art, provided that the method is capable of collecting one or more biomarkers as described herein.

In accordance with the present disclosure, the skin sample is collected from the subject by a non-invasive method. In some embodiments, the method comprises collecting one or more skin samples by a non-invasive method. In some embodiments, the method comprises non-invasively collecting one or more skin samples. In some embodiments, the methods of the invention comprise collecting one or more skin samples from a lesional area, a non-lesional area or from both using a non-invasive method.

When referring to a non-invasive method for collecting (obtaining) at least one skin sample it should be understood as a method that provides information without the need of introduction of instruments into the body, specifically, into the skin, and into the epidermis layer. For example, the non-invasive methods of the invention do not make use of needles or microneedles for collecting one or more skin samples as well as of tape striping for that purpose. In some embodiments, the methods for collecting one or more skin samples do not use needles or microneedles. In some other embodiments, the methods for collecting one or more skin samples do not use of tape stripping.

In some other embodiment, the method of the invention comprises non-invasively collecting one or more samples from the skin surface.

In some other embodiment, the method of the invention comprises non-invasively collecting one or more samples from the epidermis surface.

In some other embodiment, the method of the invention comprises non-invasively collecting one or more samples from the stratum corneum.

Hence, in accordance with some aspects, the present disclosure provides a method comprising the steps of non-invasively collecting one or more skin samples from a subject and determining at least one biomarker in the one or more skin sample. The method further comprises classifying the subject and in accordance with some embodiments, administering a treatment regimen to a subject classified as a responder.

In some embodiments, the non-invasive method comprises one or more of well extract (also denoted at times as skin wash sampling) skin auto-fluorescence, swab testing, sweat patches, Transdermal Analyses Patch, hydrogel micro patch, and swab extract.

In some other embodiments, the method of the invention is applicable using non-invasive methods for collecting one or more biomarkers. In some embodiments, the non-invasive method comprises Near IR (Infra red) and/or thermodynamic camera.

It should be noted that non-invasive methods typically do not cause inconvenience to skin and in particular to lesional area.

In some embodiments, the method comprises collecting one or more skin samples using well extract technique. A well extract as used herein refers to a non-invasive method for collecting skin extract by placing a well (e.g. 1 cm in diameter) on the skin of the subject and pressing it down followed by collecting a solution through the opening on the well. It was suggested that a well extract is suitable for collecting a skin sample comprising one or more hydrophilic biomarkers. In some embodiments, the method comprises collecting at least one skin hydrophilic biomarker secreted from the skin surface, from the epidermis surface or from the stratum corneum. In some embodiments, the method comprises collecting at least one skin hydrophilic biomarker by well extract technique. In some embodiments, the method comprises collecting at least one skin hydrophilic biomarker secreted from the skin surface, from the epidermis surface or from the stratum corneum by well extract technique.

In some embodiments, the method comprises collecting one or more skin samples using swab extract technique. A swab extract as used herein refers to a non-invasive method for collecting skin extract by holding the swab, so the shaft is parallel to the skin surface and the swab is rubbed back and forth along the surface applying firm pressure while rotating the swab head continuously. It was further suggested that a swab extract technique is suitable for collecting a skin sample comprising one or more hydrophilic biomarkers and/or one or more hydrophilic biomarkers.

As described herein, it was surprisingly findings that specific types of materials including, inter alia, inert materials (sterile and non-sterile) can be used for accurately determine biomarkers and ORAC in skin samples.

Hence, in accordance with some aspects, the present disclosure provides a method for obtaining (collecting) at least one skin sample from a subject, the method comprising contacting at least one skin region in the subject with a substrate material, wherein the substrate material exhibits inert characteristics and wherein the skin sample is suitable for characterizing at least one biomarker in the at least one skin sample of the subject.

In some examples, the substrate material may be designed (configured/suitable) to absorb (take up) a solution (liquid). In some examples, the substrate material can hold a solution at least twice it's wight, at times 4 times, at times 6 times, at times 8 times, at times 10, at times 13 times, at times 15 times, at times 17 times, at times 20 times, at times 23 times, at times 25 times of the material weight.

The substrate material in accordance with the present disclosure is not limited to a specific material and may be any a material sterile and/or a non-sterile material.

In the context of the present disclosure, the substrate material exhibits inert characteristics such that the substate material does not undergo chemical reactions or chemical/physical changes when exposed to various conditions (including environmental and/or chemical conditions). It should be noted that the term substate material as used herein encompasses one or more materials.

It should be further noted that in some examples, in which the substate material comprises one material and/or in some examples, in which the substate material comprises two materials or more, the single material or the combination of the two or more materials exhibit inert characteristics.

In other words, the substate material as described herein, either a single material or a combination of materials exhibits inert characteristics such that the substate material does not undergo an oxidation/reduction reaction (has no potential in undergoing this reaction).

Based on the results shown below, it was suggested that the substrate material that is applicable for the present disclosure can not undergo oxidation and/or reduction and is characterized by low (weak) tendency to undergo oxidation/reduction reaction.

Hence, in accordance with some aspects, the present disclosure provides a method for obtaining (collecting) at least one skin sample from a subject, the method comprising contacting at least one skin region in the subject with a substrate material, wherein the substrate material is essentially free of oxidation and/or reduction characteristics and wherein the skin sample is suitable for characterizing at least one biomarker in the at least one skin sample of the subject.

In accordance with some other aspects, the present disclosure provides a method for obtaining (collecting) at least one skin sample from a subject, the method comprising contacting at least one skin region in the subject with a substrate material, wherein the substrate material is essentially free of an oxidizing agent and/or essentially free of an antioxidant agent and wherein the skin sample is suitable for characterizing at least one biomarker in the at least one skin sample of the subject.

In some examples, the substrate material is essentially free of an antioxidant.

The term antioxidant as used herein refers to a compound/agent/substance that inhibit oxidation and hence inhibits a chemical reaction that may typically produce free radicals.

In some examples, the substrate material is characterized by low (weak) antioxidant capacity and at times is essentially free of antioxidant activity.

Without being bound by theory, it was suggested that the substrate material does not comprises an antioxidant agent. i.e. is essentially free of an antioxidant agent.

Hence, the substrate material in accordance with the present disclosure is free of any material that may have an antioxidative activity.

In some examples, the substrate material is essentially free of an antioxidative activity. The tern antioxidative activity refers to the ability of a material to exhibit antioxidant properties and is often related to the capacity of a material to neutralize free radicals.

In some examples, the substrate material is characterized by low (weak) antioxidant capacity. The term antioxidant capacity as used herein refers to ability of a material to counteract or inhibit oxidative processes, i.e. refers to the material role as an antioxidant.

The antioxidant capacity of the substrate material can be determined by any method known in the art that may assess the ability of a substrate material to neutralize or inhibit oxidative reactions.

In some examples, the antioxidant capacity of the substrate material may be determined by 2,2-Diphenyl-1-picrylhydrazyl (DPPH) assay. As appreciated, the DPPH assay involves measuring a material ability to reduce DPPH, a stable free radical.

In some examples, the antioxidant capacity of the substrate material may be determined by Ferric Reducing Antioxidant Power (FRAP) assay. As appreciated, the FRAP method measures the reduction of Fe3+ (ferric) to Fe\hat{ }2+ (ferrous) ions by antioxidants and the change in color or absorbance is used to calculate antioxidant capacity.

In some examples, the antioxidant capacity of the substrate material may be determined by Trolox Equivalent Antioxidant Capacity (TEAC) assay. As appreciated, Trolox is a water-soluble vitamin E analog and is used as a standard to assess the antioxidant capacity of a substance such that the method measures the ability of a material to scavenge free radicals compared to Trolox.

In some examples, the antioxidant capacity of the substrate material may be determined by 2,2′-Azino-bis(3-ethylbenzothiazoline-6-sulfonic acid) (ABTS) assay. As appreciated, in this assay, the ABTS radical is generated, and the antioxidant's ability to reduce it is measured by changes in color or absorbance.

In some examples, the antioxidant capacity of the substrate material may be determined by Cupric Reducing Antioxidant Capacity (CUPRAC) assay. As appreciated, the CUPRAC assay evaluates the reducing capacity of a material using copper ions as the oxidizing agent.

In some examples, the antioxidant capacity of the substrate material may be determined by a chemiluminescence assay. As appreciated, the chemiluminescence assay assesses antioxidant capacity by measuring the reduction in chemiluminescence produced by free radicals or reactive species in the presence of antioxidants.

In some examples, the antioxidant capacity of the substrate material may be determined by Oxygen Radical Absorbance Capacity (ORAC). As appreciated, ORAC, is a measure of an antioxidant capacity of a material and it quantifies the ability of a material to neutralize or “absorb” free radicals.

As shown in the examples below, ORAC measurements showed a high background value from medical swabs irrespective of the solutions used (either pre-wash or post-wash). In contrast, same measurements conducted with inert material (Q tips) did not show any background noise and were similar to the value obtained for PBS alone. Interestingly, the results in the examples below showing total protein measurements are in accordance with the ORAC data. Total protein measurements using medical swabs showed strong signal in contrast with inert material (Q tips) that showed very week background, suggesting that the inert material may be applicable for protein levels measurement.

In some examples, the substate material is characterized by an ORAC value that is essentially the same as an aqueous solution. In some examples, the substate material is characterized by an ORAC value that is essentially the same as a PBS solution.

In some examples, the substate material is characterized by an ORAC value of at most 40, at times at most 30, at times at most 20, at times at most 10, at times at most 5 as measured by ORAC assay as described for example in the examples below.

In some examples, the substate material is characterized by an ORAC value of between about-5 and about 40, at times between about-5 and about 30, between about-5 and about 20, between about-5 and about 10 between about-5 and about 5.

As described herein, the substrate material may be a sterile material (e.g. a material that undergo a sterilization process) or a non-sterile material.

In some examples, the substrate material is essentially free of an oxidant (oxidizing agent). The term oxidant as used herein refers to a compound/agent/substance in a redox chemical reaction that gains or “accepts”/“receives” an electron from another substance (denoted as a reducing agent). In other words, an oxidant is any substance that oxidizes another substance.

In some examples, the substrate material is or comprises a non-woven fiber.

The term non-woven fiber as used herein refers to a fabric-like material made from staple fibre (short) and long fibres (continuous long), bonded together by chemical, mechanical, heat or solvent treatment.

In some examples, the substrate material is or comprises a polysaccharide.

In some examples, the substrate material is or comprises cellulose. In some examples, the substrate material is or comprises pure cellulose.

In some examples, the substrate material is or comprises cotton fibers. The term cotton fiber refers to natural hollow fibers comprising mainly of cellulose. Cellulose refers to a polysaccharide consisting a linear chain of hundred to many thousands of β(1→4) linked D-glucose units.

In some examples, the substrate material is or comprises pure cotton. As used herein the term pure cotton refers to cotton fabric without any synthetic or blended materials. As described herein, the substate material may be a sterile material or a non-sterile material.

It was suggested that a sterile material in accordance with the present disclosure may undergo sterilization in an environment that is free of antioxidant agents which may adhere to the substrate material. In some examples, the substrate material may undergo sterilization in an environment that is free of one or more antioxidants. In some examples, the substrate material may undergo sterilization with gamma radiation. In some examples, the substrate material is essentially free of ethylene oxide.

In some examples, the substate material is essentially free of a functional group that may undergo oxidation. In some examples, the substate material is essentially free of an epoxide group.

As described herein, method of the invention is applicable for a skin area of the subject such that a skin sample is collected following the contacting of the substate material with at least one skin region.

In accordance with some embodiments, the methods of the invention comprise a step of collecting one or more skin samples. The method described herein involves a step of contacting the substrate material as described herein with at least one skin region.

As used herein the term “contacting” means to bring together such that the substrate material is brought together by touching at least one skin region. In the context of the present invention, the term “contacting” includes all measures or steps, which allow interaction between the substrate material and at least one skin region.

In some examples, the contacting comprises rubbing the at least one skin region with the substrate material.

The method described herein may comprise one or more steps of pre-treating the substrate material before the contacting step.

In some examples, the method comprises a hydrating the substrate material with a hydrating solution, prior to contacting with at least one skin region. Hydration may be done with any liquid, including, for example, an aqueous solution.

In some examples, hydration may be with an aqueous solution. In some examples, hydration may be with phosphate buffer saline (PBS). In some examples, hydration may be with an alcohol. In some examples, hydration may be with ethanol. In some examples, hydration may be with any one of PBS, ethanol or a combination thereof.

In some examples, hydrating of the substate material is for a time period allowing hydrating at least a part of the substrate material. In some examples, hydration is for a time period of a few seconds to few days.

In some examples, the method comprises removing excess hydrating solution from the at least partially hydrated substrate material. In some examples, removing excess solution is done prior to contacting the substate material with at least one skin region.

The skin sample can be collected from various regions in the body as described herein.

In some embodiments, the method comprises collecting at least one skin hydrophilic biomarker secreted from the skin surface, from the epidermis surface or from the stratum corneum. In some embodiments, the method comprises collecting at least one skin hydrophilic biomarker and/or at least one hydrophobic biomarker by swab extract technique. In some embodiments, the method comprises collecting at least one skin hydrophilic biomarker secreted from the skin surface, from the epidermis surface or from the stratum corneum by swab extract technique. In some embodiments, the one or more biomarkers may be secreted with sweat.

As described herein, the method comprises determining an expression of the at least one biomarker, if present, in the at least one skin samples collecting from the subject.

In some embodiments, the method determining expression of at least one biomarker in a skin sample collected from the skin surface, the epidermis surface or the stratum corneum.

As noted herein, the methods described herein involve determining one or more parameters. In some embodiments, the method comprises determining an expression of at least one biomarker in at least one skin sample collected from the subject or in a skin area of the subject.

In some embodiments, the method comprises determining at least one biomarker in at least one skin sample collected from the subject, the at least one biomarker is at least one of (i) at least one protein, (ii) at least one peptide, (iii) at least one antioxidant, (iv) at least one metabolite, (v) at least one enzyme or (vi) a combination thereof.

In some embodiments, the method comprises determining at least one protein in at least one biological sample collected from the subject. In some embodiments, the method comprises determining at least one protein in at least one skin sample collected from the subject.

In some embodiments, the method comprises determining at least one cytokine in at least one biological sample collected from the subject. In some embodiments, the method comprises determining at least one cytokine in at least one skin sample collected from the subject.

In some embodiments, the method comprises determining at least one peptide in at least one biological sample collected from the subject. In some embodiments, the method comprises determining at least one peptide in at least one skin sample collected from the subject.

In some embodiments, the method comprises determining at least antioxidant in at least one biological sample collected from the subject. In some embodiments, the method comprises determining at least antioxidant in at least one skin sample collected from the subject.

In some embodiments, the method comprises determining ORAC in at least one biological sample collected from the subject. In some embodiments, the method comprises determining ORAC in at least one skin sample collected from the subject.

In some embodiments, the method comprises determining UA in at least one biological sample collected from the subject. In some embodiments, the method comprises determining UA in at least one skin sample collected from the subject.

In some embodiments, the method comprises determining at least one metabolite in at least one biological sample collected from the subject. In some embodiments, the method comprises determining at least one metabolite in at least one skin sample collected from the subject.

In some embodiments, the method comprises determining at least one enzyme in at least one biological sample collected from the subject. In some embodiments, the method comprises determining at least one enzyme in at least one skin sample collected from the subject.

In some embodiments, the method comprises determining expression of one or more of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof in at least one skin sample.

In some embodiments, the method comprises determining expression of one or more of IL-1a, IL-8, IL-1b, ORAC or a combination thereof in at least one skin sample.

In some embodiments, the method comprises determining expression of one or more of IL-1a, IL-8, IL-1b, or a combination thereof in at least one skin sample.

As indicated herein, the at least one skin sample is collected from a subject prior to initiation of treatment.

As noted herein, determining the expression of the at least one biomarker can be done by various methods known in the art.

In some embodiments, the method comprises determining expression of at least one biomarker by auto-fluorescence technique. Skin auto-fluorescence technique is generally sone using a spectrofluorometer and measuring in vivo skin auto-fluorescence of various molecule, including, inter alia, tryptophan moieties, pepsin-digestible collagen cross-links (PDCCL), collagenase-digestible collagen cross-links (CDCCL), elastin cross-links (ECL) or any combination thereof.

In some embodiments, the method comprises determining expression of at least one cytokines by enzyme-linked immunosorbent assay (ELISA) commercial kit. Such methods may include coating ELISA plates with a cytokine-specific capture antibody and incubated overnight at RT. The plates can be washed and further treated with blocking solution. Skin samples can be then introduced into the wells, incubated and washed, followed by addition of antibody specific detection molecules.

Determining the expression of ORAC in the skin sample can be done by various methods known in the art. In some embodiments, methods for determining expression of ORAC may include use of commercial kits. For example, the ORAC Assay can be done based on the oxidation of a fluorescent probe by peroxyl radicals by way of a hydrogen atom transfer (HAT) process. Peroxyl radicals are produced by a free radical initiator AAPH [2,2′-azobis(2-amidinopropane) dihydrochloride], which quenches the fluorescent probe over time. Antioxidants present in the assay work to block the peroxyl radical oxidation of the fluorescent probe until the antioxidant activity in the sample is depleted. The remaining peroxyl radicals destroy the fluorescence of the fluorescent probe. This assay continues until completion, which means both the antioxidant's inhibition time and inhibition percentage of free radical damage is a single value. The sample antioxidant capacity correlates to the fluorescence decay curve, which is usually represented as the area under the curve (AUC). AUC is used to quantify the total peroxyl radical antioxidant activity in a sample and is compared to an antioxidant standard curve of the water-soluble vitamin E analog Trolox™.

Interestingly, and as shown herein in the examples below, the inventors have identified different expression of biomarkers determined prior to treatment initiation in population of patients classified as responders and as non-responders both in lesional area and in non-lesional area.

As shown in the Examples below, various biomarkers collected from skin samples before initiation of a treatment showed differential expression that was different in responders and in non-responders. Specifically, a lower expression of biomarkers determined before initiation of treatment was observed in subject population that was classified as responded to the treatment.

As noted herein, the methods disclosed herein involves classifying the subject.

In some embodiments, classifying the subject is based on determining if the subject is responsive to the treatment regimen.

The term “response” or “responsiveness” to a certain treatment, specifically, treatment regimen, refers to an improvement in at least one relevant clinical parameter as compared to an untreated subject diagnosed with the same pathology (e.g., the same type, stage, degree and/or classification of the pathology), or as compared to the clinical parameters of the same subject prior to treatment with the indicated medicament. The term responder as used herein, encompasses any kind of response, including a complete response, good response, partial response, mild or moderate response, low response, as well as non-resistance to the treatment.

In some embodiments, classifying the subject is based on determining if the subject is non-responsive to the treatment regimen.

The term “non responder” or “drug resistance” to treatment with a specific medicament, specifically, treatment regimen that comprise the disclosed modulators, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament. The term non-responder as used herein encompasses low or poor response, as well as no response, or drug-resistant subjects.

In some embodiments, classifying the subject is based on determining if the expression determined for at least one biomarker in at least one biological sample of the subject is any one of positive or negative with respect to a predetermined expression of the at least one biomarker or to an expression of the at least one biomarker in at least one control sample.

As described herein, the likelihood of treatment success may be determined based on a set of biomarkers determined in a skin sample prior to treatment.

In some embodiments, classifying the subject is based on determining if the measure determined for at least one clinical feature in the subject is any one of positive or negative with respect to a predetermined measure of the at least one clinical feature or to a measure of the at least one clinical feature in at least one control subject.

As shown in the Examples below, high correlations were observed between female specific features and autoimmune disease. Specifically, a 75.56% accuracy was observed between women difficulty to find treatment and menstruation symptoms and age of first period. In addition, a 73.7% accuracy was observed between women's disease severity, being moderate to severe and menstruation symptoms, age of first period and number of children.

In some embodiments, classifying the subject is based on determining if the measure determined for at least one clinical feature in the subject is the same or different with respect to a predetermined measure of the at least one clinical feature or to a measure of the at least one clinical feature in at least one control subject.

In some embodiments, classifying the subject is by comparing one or more of (i) the expression of at least one biomarker determined in at least one biological sample of the subject with a predetermined expression of the at least one biomarker or with an expression of the at least one biomarker in at least one control sample, (ii) the measure of at least one clinical feature determined from the subject with a predetermined measure of the at least clinical feature or with a measure of the at least one clinical feature in a control sample, (iii) any combinations thereof.

In some embodiments, classifying the subject is by comparing the expression of at least one biomarker from the subject with a predetermined expression of the at least one biomarker.

In some embodiments, classifying the subject is by comparing the expression of the at least one biomarker from the subject with an expression of the at least one biomarker obtained from control sample.

As used herein, the term “comparing” refers to any examination of the expression obtained in the at least one sample as detailed throughout for finding similarities or differences between at least two different samples. In accordance with the present disclosure, comparing encompasses the possibility to use a computer-based approach.

In some embodiments, classifying the subject as a responder to the treatment regimen if the expression of at least one biomarker determined in the sample obtained from the subject is above or below a predetermined expression of the at least one biomarker.

As described herein, a lower expression of biomarkers was observed in skin samples collected from lesional area of a subject population that were classified as responders.

Hence, in some embodiments, the method comprises classifying a subject based on expression of one or more of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof.

In some embodiments, the method comprises classifying a subject based on expression of one or more of IL-1α, IL-1β, IL-4, IL-31, TSLP, IL-8, CXCL2, KLK5, MMP-9, FGF 2, albumin or a combination thereof.

In some embodiments, the method comprises classifying a subject as a non-responder is the expression of at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof determined from a skin sample collected from a lesional area, is higher than a predetermined expression of the at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof respectively.

In some embodiments, the method comprises classifying a subject as a non-responder if the expression of at least one of IL-1α, IL-1β, IL-4, IL-31, TSLP, IL-8, CXCL2, KLK5, MMP-9, FGF 2, albumin or a combination thereof determined from a skin sample collected from a lesional area, is higher than a predetermined expression of the at least one of IL-1α, IL-1β, IL-4, IL-31, TSLP, IL-8, CXCL2, KLK5, MMP-9, FGF 2, albumin or a combination thereof respectively.

In some embodiments, the method comprises classifying a subject as a responder if the expression of at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof determined from a skin sample collected from a lesional area, is lower than a predetermined expression of the at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof respectively. In some particular embodiments, such drug-responsive subjects or population of subjects may be associated with good prognosis.

In some embodiments, the method comprises classifying a subject as a responder if the expression of at least one of IL-1α, IL-1β, IL-4, IL-31, TSLP, IL-8, CXCL2, KLK5, MMP-9, FGF 2, albumin or a combination thereof determined from a skin sample collected from a lesional area, is lower than a predetermined expression of the at least one of IL-1α, IL-1β, IL-4, IL-31, TSLP, IL-8, CXCL2, KLK5, MMP-9, FGF 2, albumin or a combination thereof respectively.

As described herein, a lower expression of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof was determined in skin samples collected from non-lesional area in a subject population that responded to the treatment.

In some embodiments, the method comprises classifying a subject as a non-responder if the expression of at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof determined from a skin sample collected from a non-lesional area, is higher than a predetermined expression of the at least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or a combination thereof respectively.

In some embodiments, the method comprises classifying a subject as a non-responder if the expression of at least one of IL-1a, IL-1RA, IL-10, KLK5, IL-4, IL-5, IL-22, IL-33, TLSP, MMP-9, IL-8, CXCL2, IL-1b, FGF basic, ORAC or a combination thereof determined from a skin sample collected from a non-lesional area, is higher than a predetermined expression of the at least one of IL-1a, IL-1RA, IL-10, KLK5, IL-4, IL-5, IL-22, IL-33, TLSP, MMP-9, IL-8, CXCL2, IL-1b, FGF basic, ORAC or a combination thereof respectively.

In accordance with some examples, the method comprise one or more of: (i) administering the treatment regimen to a subject classified as a responder, (ii) maintaining the treatment regimen to a subject classified as a responder, (iii) increasing the dose of the treatment in subject exhibiting a mild or poor response, or (iv) ceasing the treatment regimen for a subject classified as a non-responder or poor responder.

The present disclosure provides in accordance with some aspects, a method for assessing responsiveness of the subject to a treatment regimen and/or monitoring disease progression of the subject, the method comprising: (a) determining an expression of ORAC in at least one skin sample obtained from the subject, and (b) classifying the subject as responder to the treatment regimen if the expression of ORAC is the same of higher than a predetermined expression of ORAC or the expression of ORAC in at least one control skin sample. In some embodiments, the skin sample is obtained from a non-lesion skin tissue.

The present disclosure provides in accordance with some aspects, a method for assessing responsiveness of the subject to a treatment regimen and/or monitoring disease progression of the subject, the method comprising: (a) determining an expression of ORAC in at least one skin sample obtained from the subject, (b) classifying the subject as responder to the treatment regimen if the expression of ORAC is the same of higher than a predetermined expression of ORAC or the expression of ORAC in at least one control skin sample and (c) one or more of: (i) administering the treatment regimen to a subject classified as a responder, (ii) maintaining the treatment regimen to a subject classified as a responder, (iii) increasing the dose of the treatment in subject exhibiting a mild or poor response, or (iv) ceasing the treatment regimen for a subject classified as a non-responder or poor responder. In some embodiments, the skin sample is obtained from a non-lesion skin tissue.

It should be noted that the expression is reflected by measurement and determination of a value that is a numerical representation of a quantity of a biomarker.

When referring to an expression of at least one biomarker, such as IL1α or IL1B, being lower than a predetermined expression or expression in a control sample, it should be understood as a reduction by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 70%, at least 80%, at least 90% or more.

When referring to an expression of at least one biomarker, such as ORAC, being higher than a predetermined expression or expression in a control sample, it should be understood as an increase by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 70%, at least 80%, at least 90% or more.

As described herein, the methods of the invention determining at least one clinical feature of the subject to obtain a measure of the clinical feature.

In some embodiments, the methods of the invention comprise determining an expression of at least one biomarker in at least one skin sample collected from the subject and determining at least one clinical feature of the subject to obtain a measure of the clinical feature.

In some embodiments, classifying the subject is by comparing the expression of at least one biomarker and the measure of at least one clinical feature determined in the subject with a predetermined or control measure of the expression or the measure, respectively.

In some embodiments, classifying the subject is by comparing the measure of at least one clinical feature determined in the subject with a predetermined measure of at least one clinical feature.

In some embodiments, classifying the subject is by comparing the measure of the at least one clinical feature of the subject with a measure of the at least one clinical feature obtained from control subject(s).

In some embodiments, classifying the subject is by applying a statistical model by using one or more clinical feature of a subject.

The present disclosure is not limited to a specific clinical feature. In some embodiments, the at least one clinical feature is at least one of BMI, existence of obesity, physical activity, existence of fatty liver disease, PsA, Lipidemia, existence of a heart pathology, existence of a cardiovascular pathology, existence of an autoimmune disease, presence of a psychiatric condition, existence of renal dysfunction, smoking, alcohol abuse, COVID-19, number of hours of daily sun exposure, concomitant methotrexate treatment, line of treatment or any combination thereof.

In some embodiments, the at least one clinical feature includes information online of treatment.

As appreciated, previous treatment with biologic drugs and/or non biologic drugs may affect the outcome of biologic treatment. Hence, in some embodiments, the at least one clinical feature is line of treatment. As used herein line of treatment refers to the number of biological drugs the subject was treated with at the time point of testing for the next biological treatment. A zero value for line of treatment indicates a bio-naïve subject who was not treated before with biological treatment. A value of 1 for line of treatment indicates that the subject was previously treated with at least one biological drug.

In some embodiments, the at least one clinical feature is at least one of BMI, existence of obesity, physical activity, existence of fatty liver disease, Psoriatic arthritis (PsA), or any combination thereof.

In some embodiments, the at least one clinical feature is at least one of BMI, existence of obesity, or any combination thereof.

In some embodiments, classifying the subject is by comparing a profile of the subject determined based on at least one of expression of the at least one biomarker in the biological sample collected from the subject and/or the measure of the at least one clinical feature of the subject with a control profile obtained from control subject(s).

In some embodiments, classifying the subject is by comparing a Sum value of the subject calculated based on at least one of the expression of at least one biomarker and/or the measure of at least one clinical feature in a subject to a Sum value obtained from control subject(s).

In some embodiments, classifying the subject is by comparing a Sum value of the subject calculated based on the measure of at least one clinical feature to a Sum value obtained from control subject(s).

In some embodiments, classifying the subject is by determining one or more coefficients in a statistical model.

In some embodiments, the control profile is determined based on at least one of the expression of the at least one biomarker in the biological sample obtained from a control subject(s) and/or the measure of the at least one clinical feature obtained from a control subject(s).

As described herein, the methods of the invention refer to classifying a subject based on, for example, comparing the expression of at least one biomarker with predetermined expression of the at least one biomarker or the expression of the at least one biomarker in a control sample and/or comparing the measure of one ore more clinical features with predetermined measure of one ore more clinical features or the measure of one ore more clinical features in a control sample.

The predetermined expression and/or predetermined measure may be considered at times as a threshold value.

It should be noted that a threshold value also denoted as cutoff value refers to a value that in some embodiments of the present disclosure, meets the requirements for providing both high sensitivity (true positive rate) and high specificity (true negative rate). Sensitivity relates to the rate of identification of the responder patients (samples) as such, out of a group of samples, whereas specificity relates to the rate of correct identification of responder samples as such, out of a group of samples. It should be noted that threshold value may be also provided as control sample/s or alternatively and/or additionally, as standard curve/s that display predetermined standard values for responders, non-responders, and for subjects that display responsiveness to a certain extent (level of responsiveness, e.g., low, moderate and high).

For example, and as shown herein, the threshold values reflect the result of a statistical analysis of at least one biomarker expression and differences in pre-established populations of responder or non-responder. Pre-established populations as used herein refer to population of patients known to be responsive to a treatment of interest, or alternatively, population of patients known to be non-responsive or drug-resistant to a treatment of interest.

It should be emphasized that the nature of the invention is such that the accumulation of further patient data may improve the accuracy of the presently provided threshold values, which are usually based on ROC (Receiver Operating Characteristic) curves generated according to the patient data using analytical software program.

As noted above, in some embodiments of the present disclosure, at least one control sample may be provided and/or used by the methods discussed herein. A control sample as used herein, refers to a sample of at least one subject (a subject that is known to be a non-responder, or alternatively, known to be a responder), and in some embodiments, a mixture of more than two patients.

The methods described herein may further provide monitoring disease progression of the subject. In some embodiments, monitoring disease progression comprises the steps of (a) determining at least one of: (i) an expression of at least one biomarker in at least one biological sample obtained from the subject, (ii) a measure of at least one clinical feature of the subject or (iii) a combination thereof, (b) classifying the subject, (c) administering the treatment regimen to a subject classified as a responder, (d) repeating step (a) to determine at least one of the parameters defined in sections (i) to (iii) above, for at least one more temporally separated sample of the subject.

The method comprises in accordance with some embodiments, step (e) related to predicting and/or determining disease progression in the subject.

The method comprises in accordance with some embodiments, step (f) comprising repeating step (c).

In some embodiments, the at least one more temporally separated sample may be obtained after the initiation of at least one treatment regimen. In some embodiments, at least one sample may be obtained prior to initiation of the treatment. Thus, in some embodiments, at least one sample is taken before treatment and at least one sample is obtained after initiation of treatment.

In some other embodiments, the methods disclosed herein may be applied to subjects already treated by a treatment regimen. Hence, the term administration or administrating encompasses initiation of the treatment or maintaining the treatment.

Accordingly, the first and the second samples are obtained after the initiation of the treatment. Such monitoring may therefore provide a therapeutic tool used for improving and personalizing the treatment regimen offered to the treated subject.

As indicated above, in accordance with some embodiments of the invention, in order to assess the patient condition, or monitor the disease progression, as well as responsiveness to a certain treatment (e.g., comprising at least one proteasome inhibitor), at least two “temporally-separated” test samples must be collected from the examined patient and compared thereafter, in order to determine if there is any change or difference in the proteasome localization values between the samples. Such change may reflect a change in the responsiveness of the subject. In practice, to detect a change having more accurate predictive value, at least two “temporally-separated” test samples and preferably more, must be collected from the patient.

The at least two samples can be obtained from the same patient in different time-points or time intervals. The time interval may be a period that is considered reasonable by medical staff and modified as needed according to the specific requirements of the patient and the clinical state he or she may be in.

For example, this interval may be at least one day, at least three days, at least one week, at least two weeks, at least three weeks, at least one month, at least two months, at least three months, at least four months, at least five months, at least six months, at least one year, or even more.

The present disclosure provides prognostic methods for assessing responsiveness of a subject for a specific treatment regimen and for monitoring a disease progression in a subject. It should be noted that “prognosis”, is defined as a forecast of the future course of a disease or disease, based on medical knowledge. This highlights the major advantage of the invention, namely, the ability to assess responsiveness or drug-resistance and thereby predict progression of the disease, based on the proteasome dynamics evaluated in a cell of the prognosed subject.

As described herein, the subject is suffering from a pathological disorder. In some embodiments, the pathological disorder is manifested by changes in the skin hemostasis. In some embodiments, the pathological disorder is characterized by changes in the skin hemostasis in lesional area. In some embodiments, the pathological disorder is characterized by changes in the skin hemostasis in non-lesional area.

In some embodiments, the method of the invention may be suitable for optimizing personalized treatment regimen for a subject suffering from a pathological disorder characterized by changes in the skin hemostasis reflected in lesional area and/or in non-lesional area.

In some embodiments, the method of the invention may be suitable for optimizing personalized treatment regimen for a subject suffering from a pathological disorder characterized by changes in the skin.

In some embodiments, the pathological disorder is a chronic disease. In some embodiments, the pathological disorder is a chronic disease characterized by skin manifestations reflected in lesional area and/or in non-lesional area.

The term “chronic disease” as used herein refers to a disease that is persistent or long-lasting.

In some embodiments, the method of the invention may be suitable for optimizing personalized treatment regimen for a subject suffering from a chronic disease.

In some embodiments, the chronic disease is chronic kidney disease (CKD). CKD is known as a condition in which the kidneys are damaged and hence can not filter blood.

In some embodiments, the method of the invention may be suitable for optimizing personalized treatment regimen for a subject suffering from CKD. In some embodiments, in which the subject is suffering from CKD, the lesional skin area may be characterized by at least one of rash, dry skin, itchy skin, blisters, calcium deposit, swelling, change in skin color.

In some embodiments, the chronic disease is a chronic skin disease.

In some embodiments, the method of the invention may be suitable for optimizing personalized treatment regimen for a subject suffering from a chronic skin disease.

In some embodiments, the chronic skin disease is selected from the group consisting of eczema, psoriasis, acne, rosacea, vitiligo.

In some embodiments, the pathological disorder is an immune-related disorder. In some embodiments, the pathological disorder is an immune-related disorder characterized by skin manifestations reflected in lesional area and/or in non-lesional area.

In some embodiments, such immune-related disorder may be any one of an inflammation, infectious condition, an autoimmune disease, and a proliferative disorder.

As used herein autoimmune diseases arise from an inappropriate immune response of the body against substances and tissues normally present in the body. This may be restricted to certain organs (e.g. in autoimmune thyroiditis) or involve a particular tissue in different places (e.g. Goodpasture's disease which may affect the basement membrane in both the lung and the kidney).

Autoimmune disease include (i) direct evidence from transfer of pathogenic antibody or pathogenic T cells, (ii) indirect evidence based on reproduction of the autoimmune disease in experimental animals and (iii) circumstantial evidence from clinical clues. The treatment of autoimmune diseases is typically done by compounds that decrease the immune response.

In some embodiments, the methods of the invention may be also applicable for determining the suitability for treatment of a patient suffering from an autoimmune disease.

In some embodiments, the autoimmune disease is at least one of multiple Sclerosis (MS), inflammatory arthritis, rheumatoid arthritis (RA), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, psoriatic arthritis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, Behget's syndrome, ankylosing spondylitis, pemphigus, bullous pemphigoid, dermatitis herpetiformis, inflammatory bowel disease (IBD), ulcerative colitis and fatty liver disease.

In some embodiments, the subject suffering from at least one of Rheumatoid arthritis, Ankylosing spondylitis, Crohn's disease and Ulcerative Colitis, CKD.

According to some embodiment, the method of the invention may be used in determining the suitability for treatment of a patient suffering from autoimmune disorder is associated with changes in the skin.

Hence, the methods of the invention are applicable to subject suffering from an autoimmune disorder that affects the skin either in lesional area or non-lesional area.

According to some embodiment, the method of the invention may be used in determining the suitability for treatment of a patient suffering from an autoimmune skin disorder.

In some embodiments, the autoimmune disorder is an autoimmune skin disorder.

In some embodiments, the autoimmune skin disorder is at least one of behcet's disease, dermatitis herpetiformis, atopic dermatitis, dermatomyositis, lichen planus, linear IgA disease, lupus of the skin, scleroderma, vitiligo, pyoderma gangrenosum, pemphigoid, pemphigus, vasculitis or psoriasis.

According to some embodiment, the method of the invention may be used in determining the suitability for treatment of a patient suffering from a skin disease.

In some embodiments, the subject is suffering from a skin disorder. Hence, the present disclosure encompasses any disease/disorder/condition associated with any part/segment of the skin. The term skin disease may be interchangeably with the term dermatological disease.

The skin disease as used herein encompasses an inherent (genetic) skin disease or an acquired skin disease. In some embodiments, the skin disease is a chronic disease.

In some embodiments, the skin disorder is one or more of the following: a keratoderma, an ichthyosis, epidermolysis bullosa, pachyonychia congenita, pruritis (itch), dry skin (Xerosis), eczema (including atopic dermatitis) or burns.

In some embodiments, the skin disease relates to improper skin differentiation. Improper skin differentiation relates to disruption of a differentiation process which tend to affect one or more of the skin epidermal layers; basal, spinous, granular or stratum corneum and may result from various functional mechanisms including Ca⁺² dysregulation, keratins deformation, inflammation, collagen deconstruction. In some embodiments, improper skin differentiation may result in one or more of hyperkeratosis, acanthosis, inflammation or dysregulation of barrier function.

In some embodiments, skin disease related to improper skin differentiation include keratoderma, ichthyosis or combination thereof.

In some embodiments, the skin disorder is a keratoderma. In some embodiments, the keratoderma is at least one of a diffuse keratoderma, a focal keratoderma or a punctate keratoderma. In some embodiments, the keratoderma is an inherent (hereditary) keratoderma. A hereditary keratoderma may be caused by a gene abnormality resulting, for example, in an abnormal skin protein (keratin). In some embodiments, a diffuse hereditary palmoplantar keratoderma is at least one of Mutilating Palmoplantar keratoderma with periorificial keratotic plaques (Olmstead Syndrome), Diffuse palmoplantar keratoderma, Diffuse non-epidermolytic palmoplantar keratoderma, Diffuse epidermolytic palmoplantar keratoderma (diffuse EPPK, Vorner disease, PPK cum degenerations granulose, Progressive Palmoplantar Keratoderma (Greither disease, PPK transgrediens et progrediens), Mal de Meleda (Keratosis extremitatum hereditaria transgrediens et progrediens), PPK Mutilans Vohwinkel (mutilating keratoderma, Vohwinkel syndrome, and palmoplantar keratoderma mutilans), Palmoplantar Keratoderma with sclerodactyly (hardening and thickening of the connective tissues of the fingers and toes) (Huriez syndrome), Palmoplantar Keratoderma with peridontitis (inflammation of the gums) (Papillon-Lefevre Syndrome).

In some embodiments, the skin disorder is Harlequin Ichtyosis. Ichthyosis can also be due to a new spontaneous mutation. In some embodiments, the skin disease is itch sensation.

In some embodiments, the skin condition is pruritis. In some embodiments, the skin condition is Eczema. In some embodiments, the skin condition is a burn. In some embodiments, the skin condition is a scar formation. In some embodiments, the subject is suffering from at least one of psoriasis, eczema, dermatitis, post-herpetic neuralgia (shingles), a keratoderma, an ichthyosis, epidermolysis bullosa, pachyonychia congenita, pruritis (itch), dry skin (Xerosis), scleroderma, dermatomyositis, epidermolysis bullosa, and bullous pemphigoid or cancer.

In some embodiments, the subject is suffering from psoriasis. As know, psoriasis is an autoimmune disease characterized by raised areas of abnormal skin, including, areas that may be red, pink, or purple, dry, itchy, and scaly.

In some embodiments, the method of the invention relates to methods for assessing responsiveness of the subject suffering from a chronic disease, an autoimmune disease and/or a skin disease to a treatment regimen and/or monitoring disease progression of the subject.

As noted herein, the method of the invention relates to methods for assessing responsiveness of the subject suffering from psoriasis to a treatment regimen and/or monitoring disease progression of the subject.

As noted herein, the method of the invention relates to methods for assessing responsiveness of the subject to a treatment regimen and/or monitoring disease progression of the subject.

The present disclosure is not limited to a specific treatment regimen and is suitable for any treatment regimen suitable for treating the disorder described herein. The treatment regimen may be in accordance with some examples, a small molecule, antibody (including monoclonal antibodies), fusion protein, nucleic-acid based molecule (including antisense, CRISPER based, mRNA based, gene therapy, aptamer). In some examples, the treatment regimen includes one or more monoclonal antibodies.

In some embodiments, the treatment comprises administration of one or more of at least one chemotherapeutic agent, at least one biological therapy agent, at least one immunotherapeutic agent or any combination thereof.

In some embodiments, the treatment regimen comprises administration of one or more at least one of biological treatment.

It should be noted that the term “biological treatment”, as used herein refers to any biological material that affects different cellular pathways. Such agent may include antibodies, for example, antibodies directed to cell surface receptors participating in signaling, that may either activate or inhibit the target receptor. Such biological agent may also include any soluble receptor, cytokine, peptides or ligands.

The treatment regimen to be administered to the subject is predicted to treat the autoimmune disorder and/or the skin disorder.

In some examples, the method comprises administration of a biological treatment is or comprises at least one of TNFα inhibitor, IL-23 inhibitor, IL-17 inhibitor, IL-36 inhibitor, IL-4 inhibitor, IL-13 inhibitor, Janus kinase (JAK) inhibitor or a combination thereof.

In some embodiments, the treatment regimen comprises administration of one or more of a TNFα inhibitor. A TNF inhibitor refers to an agent that suppresses the physiologic response to tumor necrosis factor (TNF).

In some embodiments, the TNFα inhibitor is or comprises an antibody. In some embodiments, the TNFα inhibitor is or comprises a monoclonal antibody. In some embodiments, the TNFα inhibitor is or comprises a circulating receptor fusion protein. In some embodiments, the TNFα inhibitor is or comprises a small molecule.

In some embodiments, the TNFα inhibitor is or comprises at least one of Infliximab, Adalimumab, Apremilast (Otezla), Certolizumab Pegol, Golimumab, Etanercept, Hyrimoz, or any combination thereof.

Infliximab, is a chimeric monoclonal antibody, sold under the brand name Remicade. Adalimumab, is a monoclonal antibody sold under the brand name Humira. Certolizumab pegol, is a fragment of a monoclonal antibody specific to TNF-α sold under the brand name Cimzia. Golimumab is a human monoclonal antibody sold under the brand name Simponi. Etanercept, is a fusion protein sold under the brand name Enbrel. Hyrimoz is a biosimilar of Adalimumab.

In some embodiments, the treatment regimen comprises administration of one or more of an IL-23 inhibitor. An IL-23 inhibitor may modulate the immune response.

In some embodiments, the IL-23 inhibitor is or comprises at least one of Ustekinumab (Stelara), Guselkumab (Tremfya), Tildrakiszumab (Ilumya), Tildrakiszumab, Risankizumab (Skyrizi) or any combination thereof.

In some embodiments, the treatment regimen comprises administration of one or more of an IL-17 inhibitors. In some embodiments, the IL-17 inhibitor is or comprises at least one of Bimekizumab, Secukinumab (Cosentyx), Ixekizumab (Taltz), Brodalumab or any combination thereof.

In some examples, the treatment regimen comprises administration of Apremilast (Otezla), Etanercept (Enbrel), Infliximab (Remicade), Adalimumab (Humira), Ustekinumab (Stelara), Secukinumab (Cosentyx), Ixekizumab (Taltz), Guselkumab (Tremfya), Tildrakizumab (Ilumya) Certolizumab (Cimzia), or Risankizumab (Skyrizi).

In some embodiments, the treatment regimen comprises administration of one or more of an IL-36 inhibitors. In some embodiments, the IL-36 inhibitor is or comprises at least Spesolimab (Spevigo).

In some embodiments, the treatment regimen comprises administration of one or more of an IL-4 inhibitors. In some embodiments, the IL-36 inhibitor is or comprises at least Dupilumab.

In some embodiments, the treatment regimen comprises administration of one or more of an IL-13 inhibitors. In some embodiments, the IL-36 inhibitor is or comprises at least Lebrikizumab.

In some embodiments, the treatment regimen comprises administration of one or more of Janus kinase (JAK) inhibitor. In some embodiments, the Janus kinase (JAK) inhibitor is or comprises at least one of baricitinib, upadacitinib, and abrocitinib.

In some examples, the biological treatment is or comprises one or more of apremilast (Otezla), Etanercept (Enbrel), Infliximab (Remicade), Adalimumab (Humira), Ustekinumab (Stelara), Secukinumab (Cosentyx), Ixekizumab (Taltz), Guselkumab (Tremfya), Tildrakizumab (Ilumya), Certolizumab (Cimzia), Sepsolimab (Spevigo), Risankizumab (Skyrizi) or any combination thereof.

The biological treatment may be administrated by any known method in the art. These include, but are not limited to, injection (e.g., using a subcutaneous, intramuscular, intravenous, intraarterial, or intradermal injection), dermal, intranasal administration and oral administration. The amount of a biological treatment may be determined by the practitioner.

As indicated above, the methods provided by the present invention may be used for the treatment of a “pathological disorder”, specifically, a chronic disease, an autoimmune disorder or a skin disorder as specified by the invention and more specifically psoriasis. It should be noted that the terms “disease”, “disorder”, “condition” and “illness”, are equally used herein.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

The present invention also provides in accordance with some aspects a kit.

In some examples, the kit comprises means to collect at least one skin sample.

In accordance with some embodiments, the kit may comprise instructions for use of the kit in a method of collecting at least one skin sample.

The means to collect at least one skin sample encompasses means that allows non-invasive collection of at least one skin sample from a subject.

In some embodiments, the kit comprises one or more swabs configured to rub one or more skin samples, and optionally one or more solutions.

In some embodiments, the kit comprises a substrate material as described herein.

In some embodiments, the kit comprises one or more wells configured to hold one or more skin samples, and one or more solutions.

In some embodiments, the kit comprises instructions for use in a method of collecting one or more skin samples. In some examples, the instructions comprise collecting one or more skin samples from a lesional skin area. In some examples, the instructions comprise collecting one or more skin samples from a non-lesional skin area.

In some examples, the kit comprises instructions and/or means to determine the expression of at least one biomarker and/or the at least one physical measure.

In accordance with some embodiments, the kit may comprise instructions for use of the kit in a method of determining at least one biomarker from the skin sample.

In accordance with some embodiments, the kit may comprise instructions for use of the kit in a method of determining expression of at least one, at least two, at least three, at least four, at least five of IL1a, IL-1RA, Albumin, KLK5, IL-10, ORAC, MMP9, IL-8, CXCL2, IL-1b, FGF basic, IL-4, IL-5, IL-22, IL-31, TSLP or a combination thereof.

In accordance with some embodiments, the kit may comprise a calibration curve and/or one control sample.

The means to determine the expression of at least one biomarker in a skin sample comprise detecting molecule specific for the at least one biomarker.

The control sample may include samples of subjects suffering from an autoimmune disease and/or a skin disease, samples of responder's subjects (having a beneficial reaction to treatment), samples of non-responders' subjects (having a non-beneficial reaction to treatment), samples of healthy subjects.

The kits of the invention are for use in the methods of the invention as described herein.

According to some embodiments, the kit of the invention used in a method for determining the efficacy of a treatment regimen in a subject suffering from a chronic disease. According to some embodiments, the kit of the invention may be used for determining if a subject suffering from an chronic disease would respond and have beneficial response to a treatment with a therapeutic agent, optionally a biological treatment.

According to some embodiments, the kit of the invention used in a method for determining the efficacy of a treatment regimen in a subject suffering from an autoimmune disease. According to some embodiments, the kit of the invention may be used for determining if a subject suffering from an autoimmune disease would respond and have beneficial response to a treatment with a therapeutic agent, optionally a biological treatment.

According to some other embodiments, the kit of the invention used in a method for determining the efficacy of a treatment regimen in a subject suffering from a skin disease. According to some embodiments, the kit of the invention may be used for determining if a subject suffering from a skin disease would respond and have beneficial response to a treatment with a therapeutic agent, optionally a biological treatment.

In some embodiments, the kit comprises instructions for use. Such instructions comprise at least one of: (i) instructions for determining an expression of at least one biomarker in at least one skin sample obtained from a subject, (ii) instructions for determining a measure of at least one clinical feature determined from a subject, (iii) instructions for comparing the expression determined in (i) with a predetermined expression of the at least one biomarker or with an expression of the at least one biomarker in at least one control sample, (iv) instructions for comparing the measure determined in (ii) with a predetermined measure of the at least clinical feature or with a measure of the at least one clinical feature in a control sample.

The present disclosure also encompasses a computer-implemented method for processing data, configured to receiving input data, such as expression of one or more biomarkers, the input data may be received from a user interface and to analyze the input data by implementing one or more of the methods described herein, using for example machine learning algorithm. The analysis may generate output data, optionally displayed on a graphical user interface, for example, as shown in the schematic representation of FIG. 1.

Disclosed and described, it is to be understood that this invention is not limited to the particular examples, methods steps, disclosed herein as such methods steps may vary somewhat. It is to be understood that the terminology used herein is used for the purpose of describing particular embodiments only and not intended to be limiting since the scope of the present invention will be limited only by the appended claims and equivalents thereof.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

For the purpose of this invention, the term “at least one” is to be interpreted broadly to encompass one or more instances of the described feature, for example one or more biomarkers. In other words, “at least one” is not considered as limiting to a singular instance and can extend to multiple instances or combinations, for example, two, three, four etc.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the forms “a”, “an” and “the” include singular as well as plural references unless the context clearly dictates otherwise.

It should be noted that the various embodiments and examples detailed herein in connection with various aspects of the invention may be applicable to one or more aspects disclosed herein. It should be further noted that any embodiment described herein may be applied separately or in various combinations. Various embodiments and aspects of the present invention as delineated hereinabove and as claimed in the claims section below find experimental support in the following examples that form an integral part of this disclosure. The phrases “in another embodiment” or any refence made to embodiment as used herein do not necessarily refer to different embodiment, although it may. Thus, as noted herein, various embodiments of the invention can be combined (from the same or from different aspects) without departing from the scope of the invention. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention. Various embodiments and aspects of the present invention as delineated herein above and as claimed in the claims section below find experimental support in the following examples.

### NON-LIMITING EXAMPLES

### Example 1: Determining Skin Biomarkers Using Different Swabs

### Material and Methods

The experiments described below were performed with different swabs including swabs that are typically referred to as cotton swabs (denoted herein as “Q-tips”) and medical swabs typically used to collect body samples (denoted herein as “medical swabs”).

Obtaining Skin Samples from Patients

Q tip swabs (Super Pharm) was used in this experiment The head of the Q tips was inserted into a 2 ml Eppendorf vial containing 1.5 ml phosphate saline buffer (PBS) for a few seconds. Excessive PBS was then gently squeezed the onto the internal wall of the Eppendorf. The head part of the Q tips swab was used to rub the skin 20 strikes back and forth (1 strike=rubbing of the swab back and forth) along an area not bigger than 5 cm such that the rubbing was done at the maximal intensity and the Q tips head was rubbed as much as possible on the tested surface area and not just the tip of the head and not just one side of it and that swirling the swab around while rubbing the head was done to assure maximal contact between the skin and the cotton head of the swab. The tested areas were (i) at least one lesional area comprising psoriasis plaque, preferably the limbs and (ii) at least one non-lesional area being at the inner side of the forearm. In addition, samples were obtained from right non-lesional area and left non-lesional area intermittently of the patients.

After sampling, the Q tips head was inserted back into the 2 ml Eppendorf tube with the PBS solution and was fully immersed into the PBS. The Eppendorf comprising the head were kept overnight at 4° C. and in the next day were transferred to −20° C. until further analysis.

As control, the same sampling was performed with a sterile swab on the same lesional area and on the same no-lesional area.

In total four samples were obtained for each patient: one sample from a lesional area with the two types of swabs, and one sample from a non-lesional area with the two types of swabs.

### Biomarker Measurements

ELISA commercial kits were used for determining cytokines and proteins expression. Protein levels were measured by commercial BCA kit (Thermo Fisher).

ORAC measurements may include use of commercial kits. For example, the ORAC Assay can be done based on the oxidation of a fluorescent probe by peroxyl radicals by way of a hydrogen atom transfer (HAT) process. Peroxyl radicals are produced by a free radical initiator AAPH [2,2′-azobis(2-amidinopropane) dihydrochloride], which quenches the fluorescent probe over time. Antioxidants present in the assay work to block the peroxyl radical oxidation of the fluorescent probe until the antioxidant activity in the sample is depleted. The remaining peroxyl radicals destroy the fluorescence of the fluorescent probe. This assay continues until completion, which means both the antioxidant's inhibition time and inhibition percentage of free radical damage is a single value. The sample antioxidant capacity correlates to the fluorescence decay curve, which is usually represented as the area under the curve (AUC). AUC is used to quantify the total peroxyl radical antioxidant activity in a sample and is compared to an antioxidant standard.

### Results

### Example 1A: ORAC Measurements of Sterile (by Ethylene Oxide) and Non-Sterile Swabs

ORAC activity of medical swabs (sterile and non-sterile) are shown in Table 1A.

TABLE 1A
ORAC measurements using different medical swabs

| | | post-wash solution | |
|---|---|---|---|
| | Pre-wash solution | (the solution in | SMPavg. |
| Swab (sterile/ | (few seconds and | which the swab was | Eq. to uM |
| non-sterile) | stir well) | deepen in) | Trolox. |
| Sterile | no | H₂O | 45.6 |
| Sterile | no | H₂O | 77.5 |
| Non Sterile | no | H₂O | 97.9 |
| Non Sterile | no | H₂O | 173.9 |
| Sterile | no | PBS | 53.1 |
| Sterile | no | PBS | 51.6 |
| Non Sterile | no | PBS | 125.3 |
| Non Sterile | no | PBS | 65.9 |
| Sterile | H₂O | PBS | 86.0 |
| Sterile | H₂O | PBS | 125.1 |
| Non Sterile (Q Tip) | H₂O | PBS | 174.3 |
| Non Sterile (Q Tip) | H₂O | PBS | 94.7 |
| Sterile | 5% EtOH (v/v) | PBS | 211.5 |
| Sterile | 5% EtOH (v/v) | PBS | 261.1 |
| Non Sterile | 5% EtOH (v/v) | PBS | 261.6 |
| Non Sterile | 5% EtOH (v/v) | PBS | 266.5 |
| Sterile | 10% EtOH (v/v) | PBS | 42.50 |
| Sterile | 10% EtOH (v/v) | PBS | 124.9 |
| Non Sterile | 10% EtOH (v/v) | PBS | 163.2 |
| Non Sterile | 10% EtOH (v/v) | PBS | 187.9 |
| Sterile | 25% EtOH (v/v) | PBS | 148.3 |
| Sterile | 25% EtOH (v/v) | PBS | 283.3 |
| Non Sterile | 25% EtOH (v/v) | PBS | 251.1 |
| Non Sterile | 25% EtOH (v/v) | PBS | 246.6 |
| Sterile | 50% EtOH (v/v) | PBS | 196.0 |
| Sterile | 50% EtOH (v/v) | PBS | 247.2 |
| Non Sterile | 50% EtOH (v/v) | PBS | 454.1 |
| Non Sterile | 50% EtOH (v/v) | PBS | 428.9 |
| PBS | | | −2.3 |
| PBS | | | −3.6 |
| PBS | | | −4.0 |
| PBS | | | −6.4 |

As can be seen from Table 1A, the ORAC measurements that were performed on the swabs (either sterile or non-sterile) showed a high background value, irrespective of the solutions used (either pre-wash or post-wash). In other words, the high background value of the swabs was obtained using all tested solutions.

In contrast, as shown in Table 1B, the same measurements done with the Q tips did not show any background noise and the measured values were compatible with the value obtained for PBS alone.

TABLE 1B
ORAC measurements using Q tips

| | SMPavg. |
|---|---|
| Non sterile Swab (Q tips) | Eq. to uM Trolox. |
| Q-tipes 4° C. | −3.9 |
| Q-tipes 4 C. | −0.3 |
| Q-tipes 4° C. over night and | −0.2 |
| freezing at −20° C. | |
| Q-tipes 4° C. over night and | 2.8 |
| freezing at −20° C. | |

### Example 1B: Protein Measurements in Different Swabs

Protein levels were measured in samples using different swabs. The results are shown in Table 1C.

### Table 1C Shows Protein Level in Samples Solutions

| | Protein (ug/ml) | | | | | | Protein ug/ml | | |
|---|---|---|---|---|---|---|---|---|---|
| | Rep_1 | Rep_2 | Rep_3 | Rep_4 | Rep_5 | Rep_6 | Average | % RSD | STDev |
| Swab_1 | 42.7 | 43.7 | 44.0 | 42.5 | 43.7 | 43.0 | 43.2 | 1.3 | 0.6 |
| Swab_2 | 28.9 | 29.8 | 29.4 | 29.1 | 29.3 | 30.1 | 29.4 | 1.3 | 0.4 |
| Q-Tip 1 | 0.95 | 1.12 | 1.12 | 1.78 | 0.95 | 1.12 | 1.2 | 24 | 0.3 |
| Q-Tip 2 | 1.28 | 0.95 | 1.28 | 0.95 | 0.95 | 0.95 | 1.1 | 15 | 0.2 |

From the results of Table 1C it can be seen that the medical swabs were not compatible with measurements of total protein levels as they showed strong signal that may interfere to biologic signal. Contrary to that, Q-tips swabs showed a very week background, hence they may be applicable for protein levels measurement. Protein levels are important as they can assist in normalized our skin sample methodology.

Normalization of Q tips samples is shown in Table 1D that shows IL-1α levels normalized with protein level in skin samples of two healthy subjects.

Table 1D Shows IL-1α Levels Normalized with Protein Level.

| Sample | Subject | IL-1a | μg | IL-a pg/ml/ |
|---|---|---|---|---|
| # | # | pg/ml | Protein/ml | (μg protein) |
| 1 | 1 | 160.72 | 15.16 | 10.60 |
| 2 | 1 | 232.37 | 17.04 | 13.64 |
| 3 | 2 | 36.59 | 22.79 | 1.61 |
| 4 | 2 | 43.63 | 44.90 | 0.97 |

Normalized values were obtained by dividing a cytokine level by total protein levels.

### Example 2: ORAC Measurements in Skin Samples Using Different Swabs

Skin samples obtained using Ethylene oxide sterile medical swabs provided high values in ORAC measurements as can be seen from Table 2, showing results obtained in samples from healthy volunteers (with no lesions). All samples were obtained using 10 strikes back and forward and were added to a volume of 1.5 ml.

TABLE 2
ORAC measurements using sterile swab.

| | | Sample average in μM |
|---|---|---|
| | | Trolox equivalents |
| Patient No. | Area of sampling | (SMPavg) |
| #1 | Right upper forearm (outer) | 222.0 |
| #1 | Left elbow | 155.4 |
| #1 | Right elbow | 156.2 |
| #1 | Left upper forearm (outer) | 198.4 |
| #2 | Left elbow | 123.1 |
| #2 | Right elbow | 159.3 |
| #2 | Left upper forearm (outer) | 187.3 |
| #2 | Right upper forearm (outer) | 141.1 |
| #3 | Left elbow | 147.4 |
| #3 | Right elbow | 167.4 |
| #3 | Left upper forearm (outer) | 155.8 |
| #3 | Left upper forearm (outer) | 167.7 |
| #3 | Right upper forearm (outer) | 170.6 |
| #3 | Right upper forearm (outer) | 120.4 |
| Blank medical swab | Swab without sampling | 134.5 |
| Blank PBS | PBS only | 0.0 |

As can be seen from Table2, the ORAC value of the blank medical swab is as and even higher than ORAC values as determined from skin samples. The PBS blank sample did not show any signal. Hence, based on these results, it was concluded that there is a noise background and ORAC assay cannot be performed using commercial medical swabs.

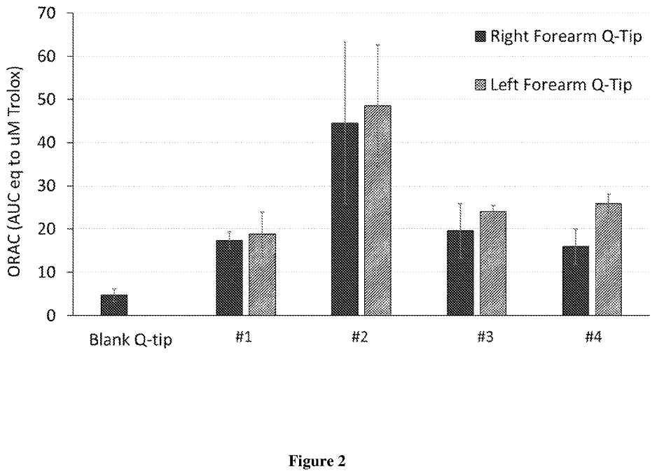

In contrast and as shown in FIG. 2, ORAC values measured in the skin samples using the Q-tips. As can be seen, the value of blank measurement was low and hence can be reduced from the measurements, suggesting that Q tips can be used as a reliable enactment for ORAC detection.

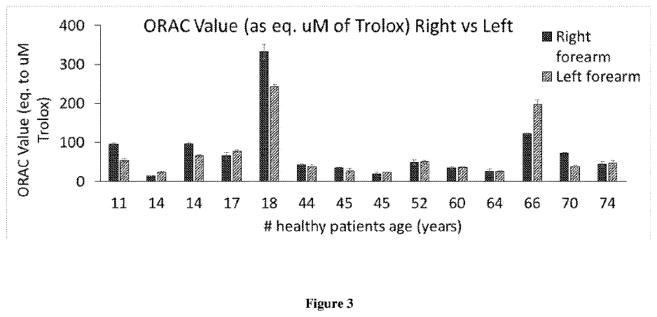

As can be seen from FIG. 3 that shows ORAC measurements in skin areas using non-sterile Q-tips swabs in healthy individuals at different ages, the measured values do not vary between the two arms and no variations were observed between healthy individuals at different ages.

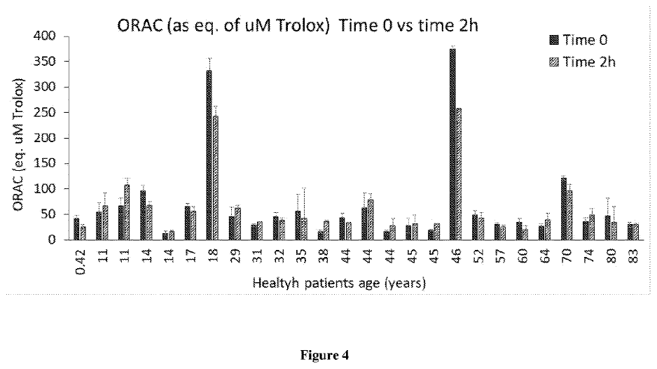

As can be seen from FIG. 4, that shows ORAC measurements in skin areas using non-sterile Q-tips swabs in healthy individuals at different ages over time, the measured values do not vary with time and no variations were observed between healthy individuals at different ages.

Taken together, these results demonstrate that the measurements of ORAC using Q-tips swabs provides repeatable results and hence can be used for collecting of skin samples.

### Example 3: Proteins and Cytokines Measurements Using Q-Tips

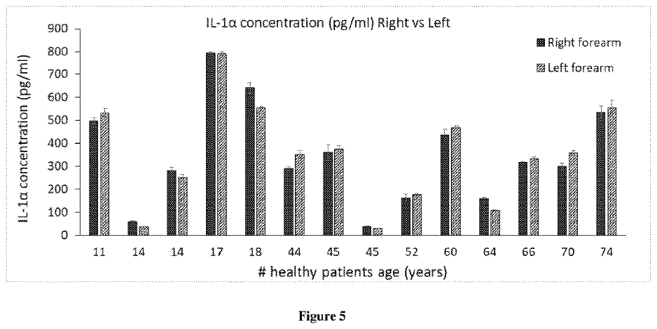

FIG. 5 shows IL-1α measurements in skin areas using non-sterile Q-tips swabs in healthy individuals at different ages. As can be seen from FIG. 5, Q tips provide reproducible results in both arms and variations were observed between healthy individuals at different ages.

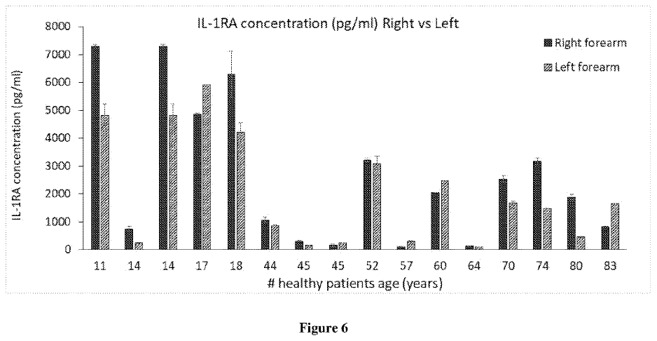

FIG. 6 shows IL-1RA measurements in skin areas using non-sterile swabs in healthy individuals. As can be seen from FIG. 6, Q tips provide reproducible results in both arms and variations were observed between healthy individuals at different ages.

FIG. 7 shows IL-1RA/IL-1α in skin areas using non-sterile Q-tips swabs in healthy individuals at differ ages. As can be seen from FIG. 7, Q tips provide reproducible results in both arms and variations were observed between healthy individuals at different ages.

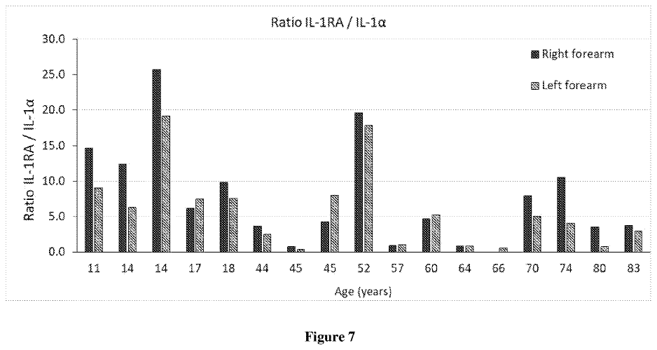

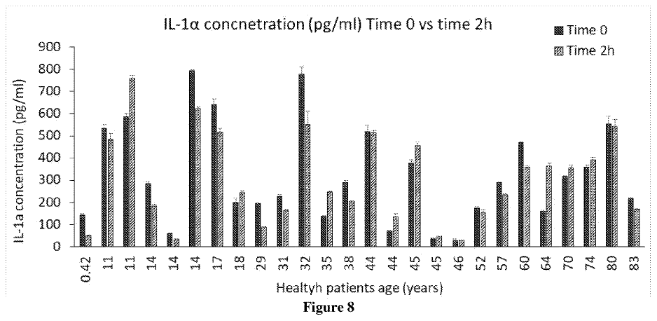

FIG. 8 shows IL-1α measurements in skin areas using non-sterile Q-tips swabs in healthy individuals at different ages over time. As can be seen from FIG. 8, Q tips provide reproducible results in both arms and variations were observed between healthy individuals at different ages.

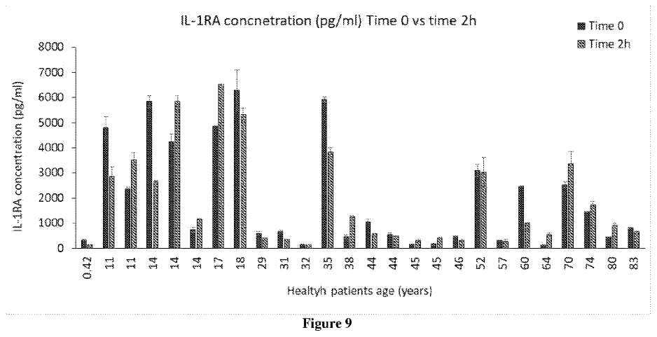

FIG. 9 shows IL-1RA measurements in skin areas using non-sterile Q-tips swabs in healthy individuals at different ages over time. As can be seen from FIG. 9, Q tips provide reproducible results in both arms and variations were observed between healthy individuals at different ages.

### Example 4: Prediction of Responsiveness Based on Skin Samples

An exemplary process that may be used to anticipate/predict the response to treatment involves, prior to prescribing treatment (either new or switching), skin samples are non-invasively collected from a patient (from lesional and/or non-lesional areas) and relevant biomarkers and/or clinical parameters are determined from these skin samples.

Through careful examination and statistical analysis, a predictive model is implemented in order to provide valuable insights into predicting how individuals may respond to specific therapeutics.

Based on the information from the predictive model, a physician prescribes the treatment with the highest success probabilities. This predictive approach holds the potential to optimize treatment strategies, offering personalized and more effective healthcare solutions tailored to individual patient profiles.

### Example 4A: Skin Sample Preparation Using Wells

A clinical study on 10 psoriatic patients was conducted in order to determine what factors might be used to predict efficacy of biological treatment prior to biological treatment. Table 3 lists the population characteristics.

TABLE 3
Study population

| Subject | Biologic Drug | Gender | age | Response to therapy |
|---|---|---|---|---|
| NS11 | Etanercept | M | 45 | Non-responder |
| NS21 | Humira | M | 65 | Excluded due to hospitalization |
| NS31 | Etanercept | M | 54 | Non-responder |
| NS41 | Etanercept | M | 45 | Responder |
| NS51 | Humira | M | 41 | Responder |
| NS61 | Stelara | M | 56 | Non-responder |
| SR23 | Cosentyx | F | 34 | Responder |
| AI21 | Humira | F | 49 | Responder |
| ZM11 | Cosentyx | M | 49 | Responder |
| AB31 | Stelara | M | 48 | Responder |

Clinical and demographic features were recorded prior to therapy.

Skin samples were collected by noninvasive extraction of the skin with a PBS solution pH=7.4. The extract was collected prior to treatment initiation by placing a well on the skin of the patient and pressing it down onto the skin. Sterile PBS was injected into the well, that was then placed in contact with skin and kept stable and sealed by a strap. After 30 minutes, the PBS solution was collected through the opening on the well, and the well is being removed. Samples were aliquoted and stored at −80° C. for further analysis such as ELISA for biomarkers. Table 4 lists the biomarkers sampled and number of patients.

TABLE 4
Number of patients for each sample.

| Biomarker | Lesional skin | Non lesional skin |
|---|---|---|
| Uric acid (nmol/well) | 9 | 9 |
| ORAC | 9 | 9 |
| IL-8 (pg/ml) | 9 | 9 |
| IL-1β (pg/ml) | 9 | 9 |
| IL-1α (pg/ml) | 9 | 9 |

For each patient, the following clinical features were recorded: weight, BMI, obesity, ethnicity, social status, sector, physical activity, sun exposure, age of disease onset, metabolic stress syndrome, fatty liver disease, renal dysfunction, diabetes, psoriatic arthritis, concomitant medication, prior systemic treatments, prior biologic treatments, other medication not related to psoriasis.

FIGS. 10A and 10B are bar graphs showing results for IL-8; IL-1β and IL-1α (FIG. 10A) and ORAC (FIG. 10B) as determined in skin samples collected prior to treatment from Lesion skin (LE) area of psoriasis patients. The psoriasis patients were classified after treatment (at least one month after initiation of treatment) as responders or as non-responders.

The results from FIG. 10A demonstrate that levels of biomarkers such as IL-8; IL-1β and IL-1α determined from skin samples obtained from lesional skin before initiation of treatment differ in patients who were classified as responders and as non-responders to biological therapy. Specifically, the level of these biomarkers was found to be higher in patients who were classified as non-responders.

In addition, the results from and FIG. 10B demonstrate that ORAC determined from skin samples obtained from lesional skin before initiation of treatment differ in patients who were classified as responders and as non-responders to biological therapy. Specifically, the ORAC value was found to be higher in patients who were classified as non-responders.

The results suggest that these biomarkers can be used as predictors for treatment efficacy and hence can be used for adjusting the treatment.

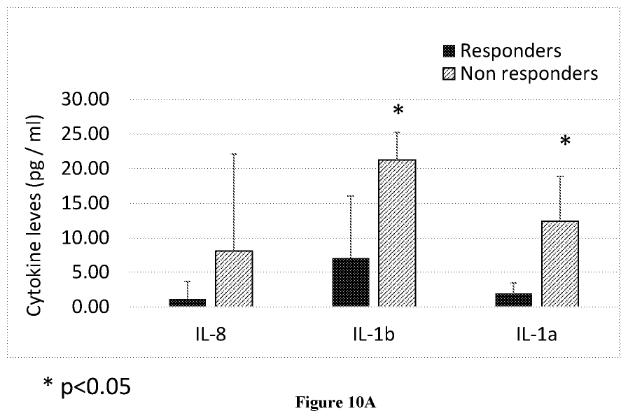

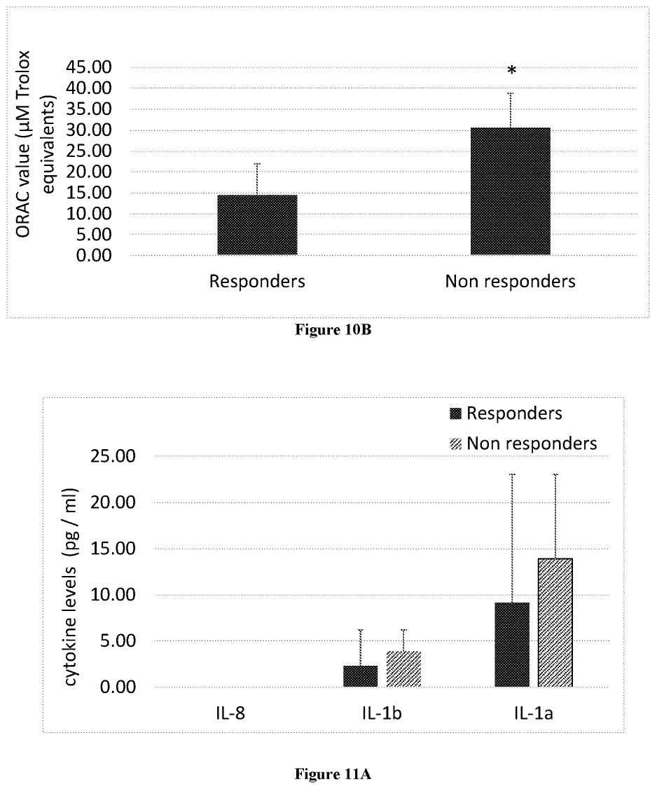

FIGS. 11A and 11B are bar graphs showing results for IL-8; IL-1β and IL-1α (FIG. 11A) and ORAC (FIG. 11B) as determined in skin samples collected prior to treatment from non-lesional skin (NL) area of psoriasis patients. The psoriasis patients were classified after treatment (at least one month after initiation of treatment) as responders or as non-responders.

The results from FIG. 11A demonstrate that no significant differences were observed in the level of the tested biomarkers as determined before initiation of treatment between patients who were classified as responders and as non-responders to biological therapy.

In addition, the results from and FIG. 11B demonstrate that ORAC determined from skin samples obtained from lesional skin before initiation of treatment are not significantly different in patients who were classified as responders and as non-responders to biological therapy.

### Example 4B: Skin Sample Preparation Using Q-Tips

A study on 18 psoriatic patients was conducted in order to determine what factors might be used to predict efficacy of biological treatment prior to biological treatment. Skin biomarkers (such as IL1a, IL-1RA, Albumin, KLK5, ORAC, MMP9, IL-8, CXCL2, IL-1b, FGF basic, IL-4, IL-5, IL-22, IL-31, TSLP) were collected non-invasively by swabs (Q-tips type) from lesional and non-lesional area psoriasis vulgaris patients (ages 25-73 years old) before initiation of treatment or before switching treatment.

Commercial ELISA kits and multiplex kits were used for quantification of interleukins and proteins and ORAC assay for total antioxidant capacity.

In addition, for each one of the patients, Psoriasis Area and Severity Index (PASI) and Physician Global Assessment (PGA) were determined prior to initiation of treatment and as described below, also after treatment for several patients.

A Pearson correlation analysis was conducted to assess the relationship between a specific biomarker and the clinical parameters, PASI and PGA. As appreciated, the Pearson correlation coefficient (r) indicate the strength and direction of the linear relationship, such that a positive value of ‘r’ would suggest a positive correlation, meaning that higher values of the biomarker are associated with higher values of the clinical parameter, while a negative value of ‘r’ would suggest a negative correlation, indicating an inverse relationship.

Tables 5A and 5B shows results of biomarkers obtained from skin samples of psoriasis patients taken from lesion skin (LE) and non-lesional skin (NL) areas using ELISA kits (Table 5A) and multiplex kits (Table 5B) and the correlation coefficient (r).

TABLE 5A
Pearson correlation between the biomarkers and clinical parameters (PASI and PGA)

| | IL-1a | IL-1RA | IL-10 | Albumin | KLK5 | IL-4 | IL-5 | IL-22 | IL-31 | TLSP |
|---|---|---|---|---|---|---|---|---|---|---|
| | pg/ml | pg/ml | pg/ml | pg/ml | pg/ml | ng/ml | pg/nl | pg/ml | pg/ml | pg/ml |
| Mean LE | 37.34 | 32359 | 3.04 | 277.16 | 2889.17 | 0.85 | 4.41 | 16.65 | 23.87 | 6.36 |
| Mean NL | 74.65 | 5316 | 2.46 | 11.43 | 991.40 | 0.63 | 5.77 | 21.85 | 28.18 | 3.09 |
| PGA LE | 0.386 | −0.018 | −0.227 | 0.333 | 0.045 | −0.277 | −0.266 | −0.240 | −0.269 | −0.272 |
| PASI LE | 0.481 | −0.187 | −0.226 | 0.063 | −0.055 | −0.237 | −0.244 | −0.228 | 0.051 | −0.145 |
| PGA NL | −0.420 | −0.003 | −0.084 | −0.320 | −0.134 | −0.004 | −0.151 | −0.083 | 0.079 | 0.067 |
| PASI NL | −0.149 | −0.134 | −0.136 | −0.096 | −0.243 | 0.045 | −0.215 | −0.144 | 0.430 | −0.047 |

TABLE 5B
Pearson correlation between the biomarkers and clinical parameters (PASI and PGA)

| | | | | | | | | ORAC | |
|---|---|---|---|---|---|---|---|---|---|
| | MMP-9 | IL-8 | CXCL2 | IL-1b | IL-1RA | IL-1a | FGF basic | eq. to μM | Protein |
| | pg/ml | pg/ml | pg/ml | pg/ml | pg/ml | pg/ml | pg/ml | Trolox | (μg/ml) |
| Mean LE | 3610.19 | 524.35 | 27.71 | 43.63 | 16767.53 | 35.46 | 1.65 | 40.37 | 42.65 |
| Mean NL | 92.00 | 23.12 | 8.72 | 7.17 | 4843.16 | 103.45 | 0.27 | 20.03 | 31.21 |
| PGA LE | 0.057 | 0.018 | 0.004 | 0.016 | −0.157 | −0.120 | 0.031 | −0.140 | 0.077 |
| PASI LE | −0.223 | −0.233 | −0.249 | −0.223 | −0.279 | 0.011 | −0.249 | −0.296 | 0.018 |
| PGA NL | −0.111 | −0.025 | −0.135 | −0.374 | −0.157 | −0.557 | −0.436 | −0.066 | 0.530 |
| PASI NL | −0.067 | −0.083 | −0.040 | −0.123 | −0.216 | −0.291 | −0.296 | −0.205 | 0.554 |

The analysis in Table 5A and Table 5B revealed a linear correlation (positive or negative) between the tested biomarkers and the clinical parameters. This suggests that these parameters are linked to the clinical status and disease progression. The findings imply that these biomarkers may be applicable in predicting responsiveness to treatments.

The values of the biomarkers determined prior to treatment initiation were also used to determine correlation to treatment responsive.

The treatment included Skyrizi (for six patients), Tremfya (for four patients), Stelara (one patient), Humira biosimilars (two patients), Humira (one patient), Taltz (one patient) and a combination of Tremfya and Taltz (one patient).

Five responders and two non-responders were examined for these biomarkers.

Table 6A and Table 6B shows results of biomarkers in responders and non-responders with samples taken from lesion skin (LE) using ELISA kits and multiplex kits, respectively.

TABLE 6A
Differences in biomarkers expression in
skin samples obtained from lesion skin.

| | IL-1a | Albumin | KLK5 | IL-4 | IL-22 | IL-31 | TLSP |
|---|---|---|---|---|---|---|---|
| | pg/ml | pg/ml | pg/ml | ng/ml | pg/ml | pg/ml | pg/ml |
| mean | 22.74 | 294 | 2663 | 0.86 | 24.66 | 32.80 | 5.48 |
| responders | | | | | | | |
| mean non- | 42.35 | 137 | 3192 | 1.98 | 27.65 | 57.08 | 23.11 |
| responders | | | | | | | |
| ratio non- | 1.86 | 0.47 | 1.20 | 2.32 | 1.12 | 1.74 | 4.22 |
| responders vs | | | | | | | |
| responders | | | | | | | |

TABLE 6B
Differences in biomarkers expression in skin samples obtained from lesion skin.

| | | | | | | FGF | ORAC |
|---|---|---|---|---|---|---|---|
| | MMP-9 | IL-8 | CXCL2 | IL-1b | IL-1a | basic | eq. to μM |
| | pg/ml | pg/ml | pg/ml | pg/ml | pg/ml | pg/ml | Trolox |
| mean | 2228 | 124 | 17.64 | 22.65 | 16.38 | 1.17 | 7.42 |
| responders | | | | | | | |
| mean non- | 3790 | 342 | 21.45 | 44.53 | 39.37 | 1.40 | 8.25 |
| responders | | | | | | | |
| ratio non- | 1.70 | 2.76 | 1.22 | 1.97 | 2.40 | 1.19 | 1.11 |
| responders vs | | | | | | | |
| responders | | | | | | | |

The results from Table 6A and Table 6B demonstrate that in addition to the biomarkers shown in FIG. 9A (IL-8; IL-1β and IL-1α), additional biomarkers (Albumin, IL-4, IL-31, TLSP, MMP-9, IL-8, IL-1b) obtained from lesional skin differ between responders and non-responders to biological therapy, with the levels of these biomarkers obtained from lesional area being higher (increased) in non-responders. This is in line with the results presented in FIG. 10A.

Table 7A and Table 7B shows results of biomarkers in responders and non-responders with samples taken from non-lesion skin (NL) using ELISA kits and multiplex kits, respectively.

TABLE 7A
Differences in biomarkers expression in skin
samples obtained from non lesion skin.

| | IL-1RA | IL-10 | IL-4 | IL-22 | | TLSP |
|---|---|---|---|---|---|---|
| | pg/ml | pg/ml | ng/ml | pg/ml | IL-31 | pg/ml |
| mean | 0.76 | 3.07 | 0.49 | 25.88 | 36.83 | 5.48 |
| responders | | | | | | |
| mean non- | 1.27 | 5.76 | 2.43 | 60.39 | 80.46 | 23.11 |
| responders | | | | | | |
| ratio non- | 1.67 | 1.88 | 4.98 | 2.33 | 2.18 | 4.22 |
| responders vs | | | | | | |
| responders | | | | | | |

TABLE 7B
Differences in biomarkers expression in skin
samples obtained from non lesion skin.

| | | | | | | | ORAC |
|---|---|---|---|---|---|---|---|
| | MMP-9 | IL-8 | CXCL2 | IL-1b | IL-1RA | IL-1a | eq. to μM |
| | pg/ml | pg/ml | pg/ml | pg/ml | pg/ml | pg/ml | Trolox |
| mean | 0.00 | 0.00 | 2.77 | 3.43 | 559 | 57.03 | 8.39 |
| responders | | | | | | | |
| mean non- | 124.82 | 8.22 | 7.80 | 0.00 | 1203 | 65.52 | 6.70 |
| responders | | | | | | | |
| ratio non- | | | 2.81 | 0.00 | 2.15 | 1.15 | 0.80 |
| responders vs | | | | | | | |
| responders | | | | | | | |

The results from Table 7A and Table 7B demonstrate that the biomarkers obtained from non-lesional skin differ between responders and non-responders to biological therapy, with the levels of these biomarkers (aside of TLSP) being higher (increased) in non-responders. This is in line with the results presented in FIG. 10A.

Taken together, these results show that the above tested biomarkers can be used to differentiate between responders and non-responders in lesional and in non-lesional areas. These results support the role of skin biomarkers measured non-invasively as a predictor for response to treatment.

In addition, Psoriasis Area and Severity Index (PASI) and Physician Global Assessment (PGA) were calculated for the two non-responder patients and for one of the responder patients. Table 8 shows the analysis in these two patients.

TABLE 8
PASI and PGA scores in non-responder patients

| | PGA | PASI | PGA | PASI | delta | delta |
|---|---|---|---|---|---|---|
| | 1st | 1st | 2nd | 2nd | PGA | PASI |
| Non-responder 1 | 3 | 12 | 3 | 12 | 0 | 0 |
| Non-responder 2 | 2 | 5.7 | 4 | 13.2 | −2 | −7.5 |
| Responder | 3 | 11.4 | 1 | 1 | 2 | 10.4 |

As can be seen in Table 8, in the two tested non-responder patients (not bionaive), no improvement and even worsening was observed in the clinical features PASI and PGA, whereas in the responder patient (bionaive) an improvement in the clinical features PASI and PGA was observed.

### Example 5: Proteins and Cytokines Measurements Using Non-Sterile Swabs in Patients Diagnosed with Psoriasis

Skin samples were obtained as described above using Q-tips from lesional and non-lesional skin areas.

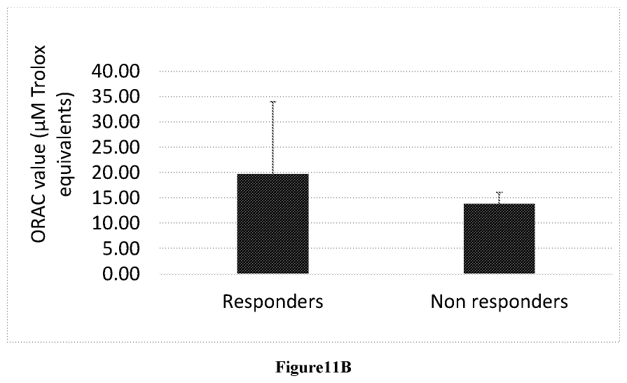

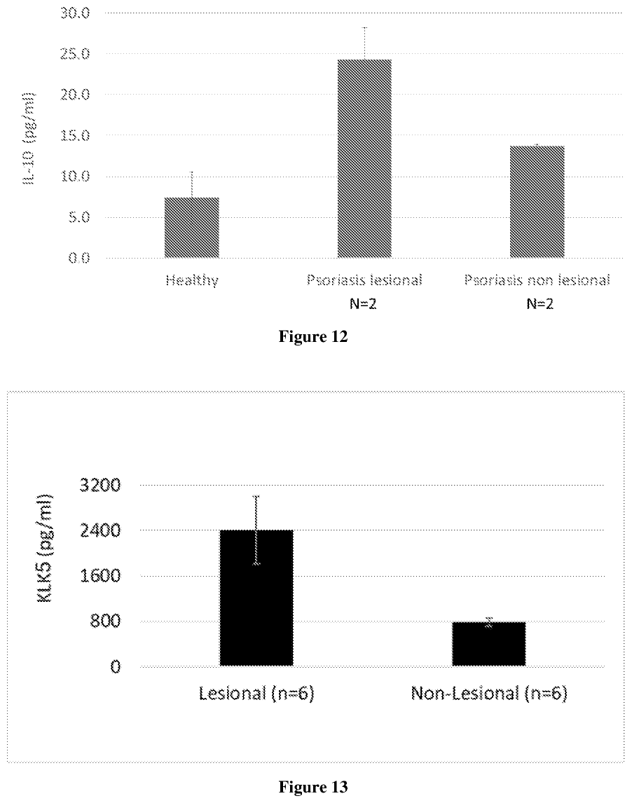

FIG. 12 shows IL-10 expression in lesions regions and in non-lesional regions of psoriasis patients as well as in healthy individuals. As can be seen in FIG. 12, expression of IL-10 was lower in healthy individuals as compared to psoriasis patients. In addition, IL-10 expression was higher in lesional area as compared to non-lesional area.

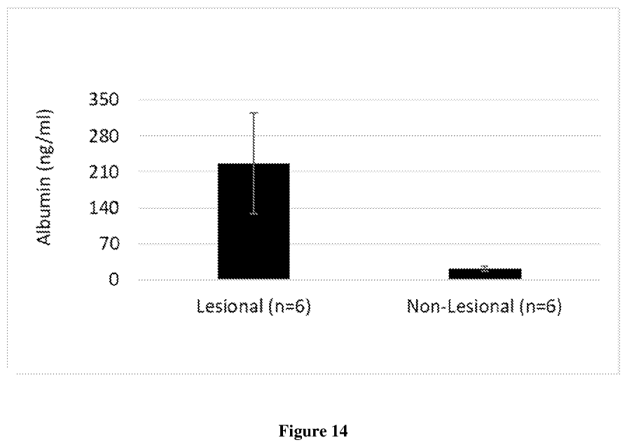

The results in FIG. 13 and FIG. 14 show KLK5 and albumin levels, respectively in lesional and non-lesional skin samples of psoriasis patients. As can be seen from FIG. 13 and FIG. 14, KLK5 and albumin levels were higher in in lesional area as compared to non-lesional area.

This suggests that non-sterile Q-tips swabs can be used to differentiate between healthy individuals and psoriasis patients as well as between lesional and non-lesional regions of psoriasis patients. In addition, these results show that the Q-tips measurements provide means to distinguish between lesional and non lesional areas in psoriasis.

### Example 6: Proteins and Cytokines Measurements Using Non-Sterile Swabs in Patients Diagnosed with Atopic Dermatitis (AD)

Skin samples were obtained as described above using Q-tips from lesional and non-lesional skin areas.

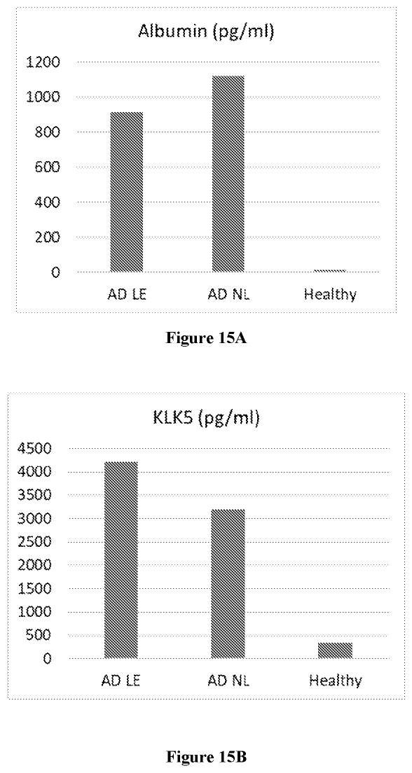

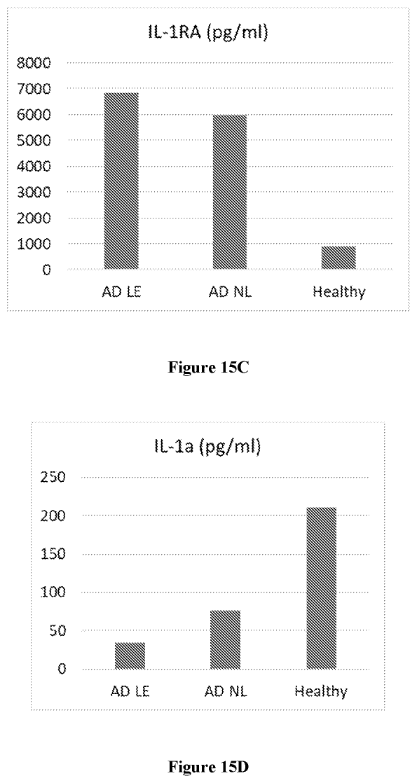

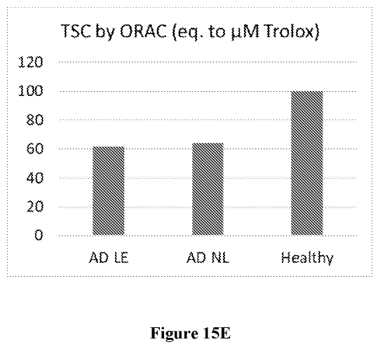

FIGS. 15A-15E show expression of various biomarkers in healthy individuals as well as in lesions regions and in non-lesional regions of AD patients.

FIG. 15A shows albumin expression in lesions regions and in non-lesional regions of AD patients as well as in healthy individuals. As can be seen in FIG. 15A, expression of albumin was lower in healthy individuals as compared to AD patients. In addition, as can be seen in FIG. 15A, expression of albumin was higher in non-lesional area.

FIG. 15B shows KLK5 expression in lesions regions and in non-lesional regions of AD patients as well as in healthy individuals. As can be seen in FIG. 15B, expression of KLK5 was lower in healthy individuals as compared to AD patients. In addition, as can be seen in FIG. 15B, expression of KLK5 was higher in lesional area as compared to non-lesional area, similar to the data shown in FIG. 12.

FIG. 15C shows IL-1RA expression in lesions regions and in non-lesional regions of AD patients as well as in healthy individuals. As can be seen in FIG. 15C, expression of IL-1RA was lower in healthy individuals as compared to AD patients. In addition, as can be seen in FIG. 15C, expression of IL-1RA was higher in lesional area as compared to non-lesional area.

FIG. 15D shows IL-1α expression in lesions regions and in non-lesional regions of AD patients as well as in healthy individuals. As can be seen in FIG. 15D, expression of IL-1α was higher in healthy individuals as compared to AD patients. In addition, as can be seen in FIG. 15D, expression of IL-1α was higher in non-lesional area as compared to lesional area.

FIG. 15E shows ORAC value in lesions regions and in non-lesional regions of AD patients as well as in healthy individuals. As can be seen in FIG. 15E, ORAC value was higher in healthy individuals as compared to AD patients. In addition, as can be seen in FIG. 15E, ORAC value was the same in non-lesional area and in lesional area.

These results suggest that non-sterile Q-tips swabs can be used to differentiate between healthy individuals and AD patients as well as between lesional and non-lesional regions of AD patients.

### Example 7: Prediction of Responsiveness Based on Skin Samples and Subject Physical Information

Integration of clinical features with skin wash biomarkers from Tables 3 and 4 was performed using linear regression analysis in a statistical model. Table 9 summarizes the linear regression analyses.

TABLE 9
Coefficients from logistic regression analysis
of skin biomarkers and clinical features:

| Variable | Coefficients |
|---|---|
| Lesional IL-1α | 0.182*** |
| Lesional IL-1β | 0.0312** |
| Obesity & IL-1β | 0.187*** |
| Obesity & IL -1α | 0.186*** |
| BMI | 0.141*** |
| Constant | −1.976** |
| AIC | −3.241 |

***p < 0.01,
**p < 0.05,
AIC = −3.241

The logistic regression model was generated using statistical tools as follows:
1. Analysis was done with glm function in stata
2. Interactions have been considered when applicable.
3. Glm executes logistic regression with logit link function—the probability of an individual responding is given by

$$g ( x ) = \frac{{e}^{x}}{1 + {e}^{x}}$$

where x represent skin biomarkers and clinical features included in the model.

As can be seen from this regression model, the combination of the two biomarkers (IL-1α and IL-1β) with physical measures, such as obesity and BMI provide an accurate prediction of the treatment response of biologic therapy in psoriatic patients.

Further, combination of biomarkers lesional levels with physical measure can be used for predicting response to biological therapy.

### Example 8: Correlation Between Women's Health and Autoimmune Disease

This example is aimed at defining the linkage between women's health and autoimmune diseases.

### Methods

Women diagnosed with an autoimmune disease were required to review and respond to the following questionnaire:
1. Are you over 18 and afflicted by an autoimmune disease?*
   Yes
   No
2. Please mark the disease you suffer from (you can mark more than one answer)?
   Lupus
   Psoriasis
   Psoriatic arthritis
   Atopic dermatitis
   Rheumatoid arthritis
   Multiple sclerosis
   Crohn's
   Colitis
   Hashimoto
   Grave's disease
   Antiphospholipid antibody syndrome (APLA)
   Celiac disease
   Type 1 diabetes
   Vitiligo
   Alopecia
   Scleroderma
   Other______
      * If you select more than one disease, please answer the following questions for the more primary disease (most severe)
3. How old are you?
4. What is your ethnicity?
   Asian
   Mediterranean
   Black\ African & Caribbean
   Middle eastern
   White\Caucasian
   Do no want to disclose
   Other______
5. Do you smoke?
   Yes
   No
   I used to
6. At what age was your disease diagnosed?______
7. What is the degree of severity of your illness as of today?
   Mild
   Mild to moderate
   Moderate to severe
   Severe
8. Have you had any disease-related skin lesions, rashes or other symptoms?
   Yes
   No
9. Are you affected by or have you experienced one of the following symptoms/diseases (you can mark more than one answer)?
   Anemia
   Endometriosis
   Cervical cancer
   Breast cancer.
   None of the above
10. Are you taking medication for your autoimmune disease?
   Yes
   No
11. Why are you not being treated:
   I am in remission
   My physicians have not found the right treatment for me
15. To what extent does the treatment alleviate the symptoms of the disease?
   Little or no noticeable effect on symptoms
   Slight to moderate
   Moderately alleviates symptoms
   Significantly
16. Did you struggle to find the right treatment for your disease? Please select the answer that best applies to you
   I am very satisfied with the first treatment that was prescribed to me
   I am being treated but some of my symptoms still persist, I wish my physician could find a better treatment
   I had to try up to 2 different treatments before I found the one that works for me
   I had to try 3 or more different treatments before I found the one that works for me
   I am still struggling to find the right medication
17. Is it a biological treatment?
   Yes
   No
18. Please state the name of the biological drug you are using______
19. Please indicate the number of years you have been treated with this biological drug
   Up to 1 year
   Between 1-3 years
   Between 3-5 years
   Over 5 years
20. At what age did you get your first period (if applicable)?______
21. If your period has stopped at what age did it stop?______
22. If you have children, how many do you have?______
23. Have you had miscarriages in the past?
   Yes—repeated miscarriages
   Yes—one miscarriage
   No
24. Have you undergone fertility treatments?
   Yes
   No
25. Is your period regular\used to be regular?
   Yes
   No
26. Have you experienced often the following symptoms before or during menstruation? (You can mark more than one answer)
   Heavy bleeding
   Premenstrual syndrome (PMS)
   Abdominal pain
   Headaches
   Breast tenderness
   Mood swings/depression
   Tiredness
   Digestive system problems
   Worsening of your autoimmune disease
   Other______
   I do not suffer from any symptoms
27. To what extent is your normal routine and performance adversely affected before or during menstruation??
   Not at all
   Slightly
   Very much
   Significantly
      Do you use hormonal supplements?
   I use hormonal contraceptives
   I used hormonal contraceptives in the past
   I use hormone replacements to relieve symptoms of menopause
   No
22. What symptoms of menopause have you experienced (you can mark more than one answer)?
   I do not suffer from any symptoms
   Heat waves
   Headaches
   Insomnia
   Mood swings/nervousness/depression/anxiety
   Joint or muscle pain
   Calcium leakage (osteopenia or osteoporosis)
   Vaginal dryness
   Dry/itchy skin
   Indigestion/heartburn
   Hair Loss
   Dizziness
   Worsening of your autoimmune disease
   Other______
   Comments
      Cramér's V analysis and Pearson correlation analysis were used to determine how strongly two categorical fields are associated. In addition, a Random Forest classifier analysis was used to determine the importance of the evaluated features.

### Results

The results of the Cramer's V analysis and of the Pearson correlation analysis based on 100 women responses to questionnaire, are shown in Table 10 and Table 11, respectively.

TABLE 10
Cramér's V analysis

| | | Cramér's |
|---|---|---|
| Feature 1 | Feature 2 | phi |
| Menopause symptoms | No. of years treating with | 0.5384878 |
| | biologics | |
| Colitis | Menopause symptoms | 0.5179045 |
| Age of first period | Psoriasis | 0.5611855 |
| Biologics name | Vaginal dryness | 0.4370037 |
| worsening autoimmune disease | Treatment efficay by | 0.4586889 |
| symptoms during menstruation | symptoms alliviation | |
| Miscarriages | No. of years treating with | 0.4698912 |
| | biologics | |
| Menopause symptoms | APLA | 0.4920419 |
| Women comorbidities | Chrohn's | 0.4333982 |
| Age of first period | Psoriasis arthritis | 0.4709476 |
| Age when period stopped | Psoriasis arthritis | 0.420084 |
| Age when period stopped | Biological treatment | 0.4516053 |
| Age when period stopped | No. of autoimmune disease | 0.3232225 |
| Menstruation symptoms | Autoimmune diseases | 0.3417574 |
| Menstruation symptoms | Disease severity | 0.3361115 |
| Disease severity | Worsening autoimmune | 0.3009858 |
| | disease symptoms during | |
| | menstruation | |
| Menopause symptoms | Chron's | 0.3864224 |

TABLE 11
Pearson correlation analysis

| | | Pearson correlation |
|---|---|---|
| Feature 1 | Feature 2 | coefficient |
| No. of women | Chrohn's | 0.4544332 |
| comorbidities | | |
| Psoriasis arthritis | Menopause symptoms: | 0.4494762 |
| | indigestion/heartburn | |

Random Forest analysis questions such as (1) women difficulty to find treatment and (2) women's disease severity, being moderate to severe, revealed the that within the top five important features, in response to (1) two feature and to (2) three features were found to be gender specific.

Specifically, the response to question (1), with a 75.56% accuracy, features #4 and #5 were number of menstruation symptoms and age of first period, respectively. In response to question (2) with a 73.7% accuracy, features #3, #4 and #5 were menstruation symptoms, age of first period and number of children, respectively.

The identification of these important gender specific features illuminates the key variables that significantly contribute to predicting the severity of the disease. This insight not only may enhance the understanding of the disease's dynamics but also provides valuable information for potential interventions or targeted investigations into the mechanisms underlying disease severity and treatment selection.

1. A method for treating for a subject suffering from a skin disease, the method comprising
(a) determining an expression of at least one biomarker selected from the group consisting of IL-1a, IL-1RA, IL-10, Albumin, Kallikrein (KLK) protein family (e.g. KLK5), IL-4, IL-5, IL-22, IL-33, TLSP, Matrix metalloproteinases (MMPs) protein family (e.g. MMP-9), IL-8, The chemokine (C—X—C motif) ligand family (e.g. CXCL2), IL-1b, IL-6 FGF basic, ORAC, GSH, UA or any combination thereof in at least one skin sample collected from said subject or in a skin area of said subject,
(b) calculating a probability of treatment success for one or more treatment regimens, based on the expression of the at least one biomarker
(c) classifying the subject as a responder or non-responder to the treatment regimen based on said calculated probability; and
(d) one or more of: (i) administering the treatment regimen to a subject classified as a responder, (ii) maintaining the treatment regimen to a subject classified as a responder, (iii) increasing the dose of the treatment in subject exhibiting a mild or poor response, or (iv) ceasing the treatment regimen for a subject classified as a non-responder or poor responder.

2. (canceled)

3. (canceled)

4. The method of claim 1, wherein classifying said subject is by comparing (i) the expression of at least one biomarker with a predetermined expression of said at least one biomarker or with an expression of said at least one biomarker in a control sample; (ii) calculating the sum of at least one expression of said at least one biomarker to obtain a Sum value; or (iii) a combination thereof.

5. The method of claim 1, comprising prior to said determining, collecting said at least one skin sample from at least one skin region, wherein said skin region is a lesional skin area and/or a non-lesional skin area.

6. (canceled)

7. (canceled)

8. The method of claim 1, comprising collecting at least one skin sample prior to initiation of said treatment regimen.

9. The method of claim 1, wherein said at least one skin sample comprises at least one biomarker secreted from the epidermis.

10. (canceled)

11. (canceled)

12. (canceled)

13. The method of claim 1, wherein said at least one biomarker is or comprises at least one of L-1α, IL-1β, IL-4, IL-31, TSLP, IL-8, CXCL2, KLK5, MMP-9, FGF 2, albumin or a combination thereof.

14. The method claim 1, wherein a subject is classified as a responder to said treatment regimen if the expression of at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof is lower than a predetermined expression of the at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof respectively or to an expression of at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof respectively in at least one control skin sample, or
a subject is classified as a non-responder to said treatment regimen if the expression of at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof is higher than a predetermined expression of the at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof respectively or to an expression of at least one of least one of IL-1a, IL-1RA, IL-10, Albumin, KLK5, IL-4, IL-5, IL-22, IL-33, TLSP, MMP-9, IL-8, CXCL2, IL-1b, FGF basic, ORAC or any combination thereof respectively in at least one control skin sample.

15. (canceled)

16. The method of claim 5, wherein said collecting said at least one skin sample is by at least one of well extract, skin auto-fluorescence, swab testing, sweat patches, transdermal analyses patch, hydrogel micro patch, and swab extract.

17. (canceled)

18. The method of claim 5, wherein said collecting said at least one skin sample is by comprising contacting at least one skin region with a substrate material.

19. The method of claim 18, wherein said substrate material (i) exhibits inert characteristics, (ii) is essentially free of oxidation and/or reduction characteristics, (iii) is essentially free of an oxidizing agent and/or essentially free of an antioxidant agent, (iv) is essentially free of an antioxidant.

20. (canceled)

21. (canceled)

22. (canceled)

23. The method of claim 18, wherein said material is characterized by an ORAC value of between about-5 and about 40.

24. The method of claim 18, wherein said substrate material is or comprises (i) a non-woven fiber and/or (ii) a polysaccharide.

25. (canceled)

26. The method of claim 18, wherein said substrate material is or comprises cellulose, optionally said substrate material is or comprises cotton fibers.

27. (canceled)

28. The method claim 1, comprising determining at least one clinical feature in said subject, optionally wherein said at least one clinical feature is at least one of BMI, existence of obesity, physical activity, existence of fatty liver disease, PsA, Lipidemia, existence of a heart pathology, existence of a cardiovascular pathology, existence of an autoimmune disease, presence of a psychiatric condition, existence of renal dysfunction, smoking, alcohol abuse, COVID-19, number of hours of daily sun exposure, concomitant methotrexate treatment, line of treatment or any combination thereof.

29. (canceled)

30. (canceled)

31. (canceled)

32. (canceled)

33. (canceled)

34. (canceled)

35. (canceled)

36. The method of claim 1, wherein said subject is suffering from psoriasis or atopic dermatitis.

37. (canceled)

38. The method of claim 1, wherein said treatment regimen comprises at least one biological treatment.

39. The method of claim 38, wherein said biological treatment is or comprises at least one of TNFα inhibitor, IL-23 inhibitor, IL-17 inhibitor, IL-36 inhibitor, IL-4 inhibitor, IL-13 inhibitor, Janus kinase (JAK) inhibitor or a combination.

40. The method of claim 39, wherein said biological treatment is or comprises one or more of apremilast (Otezla), Etanercept (Enbrel), Infliximab (Remicade), Adalimumab (Humira), Ustekinumab (Stelara), Secukinumab (Cosentyx), Ixekizumab (Taltz), Guselkumab (Tremfya), Tildrakizumab (Ilumya), Certolizumab (Cimzia), Sepsolimab (Spevigo), Risankizumab (Skyrizi) or any combination thereof.

41. (canceled)

42. A kit for determining the efficacy of a treatment regimen in a subject suffering from a skin disease, the kit comprises (i) one or more wells configured to hold one or more skin samples and/or a substrate material for collecting at least one skin sample from the subject, wherein said substrate material is inert and essentially free of oxidation and/or reduction characteristics and (ii) means to determine the expression of at least one biomarker in said skin sample and/or the at least one physical measure and optionally instructions of collecting at least one skin sample from a subject and characterizing at least one biomarker in said at least one skin sample of said subject.

43. (canceled)

44. (canceled)

45. (canceled)

46. (canceled)

47. A method for collecting at least one skin sample from a subject, the method comprising contacting at least one skin region in said subject with a substrate material, wherein said substrate material is inert and is essentially free of oxidation and/or reduction characteristics; and wherein said skin sample is suitable for characterizing at least one biomarker in said at least one skin sample of said subject.

48.-63. (canceled)
